(19) United States
(12) Patent Application Publication
(10) Pub. No.: US 2025/0092069 A1
(43) Pub. Date: Mar. 20, 2025

(54) SILYL-LIPID CANNABINOIDS WITH ENHANCED BIOLOGICAL ACTIVITY

(71) Applicant: The Regents of the University of California, Oakland, CA (US)

(72) Inventors: Annaliese Franz, Oakland, CA (US); Angel Cobo, Oakland, CA (US)

(21) Appl. No.: 18/832,711

(22) PCT Filed: Jan. 25, 2023
(86) PCT No.: PCT/US2023/011539

§ 371 Date: Jul. 24, 2024

Related U.S. Application Data
(60) Provisional application No. 63/302,723, filed on Jan. 25, 2022.

(51) Int. Cl. C07F 7/08; A61K 31/695; A61P 25/28

(52) U.S. Cl. CPC C07F 7/081; A61K 31/695; A61P 25/28; C07F 7/0812; C07F 7/0816; C07F 7/0827

(57) ABSTRACT

Provided herein are silyl lipid compounds that are cannabinoid analogs. The provided compounds are particularly useful for treating neurodegenerative disorders, and for alleviating symptoms associated with epilepsy and inflammation. Also provided are compositions and methods including the provided compounds.

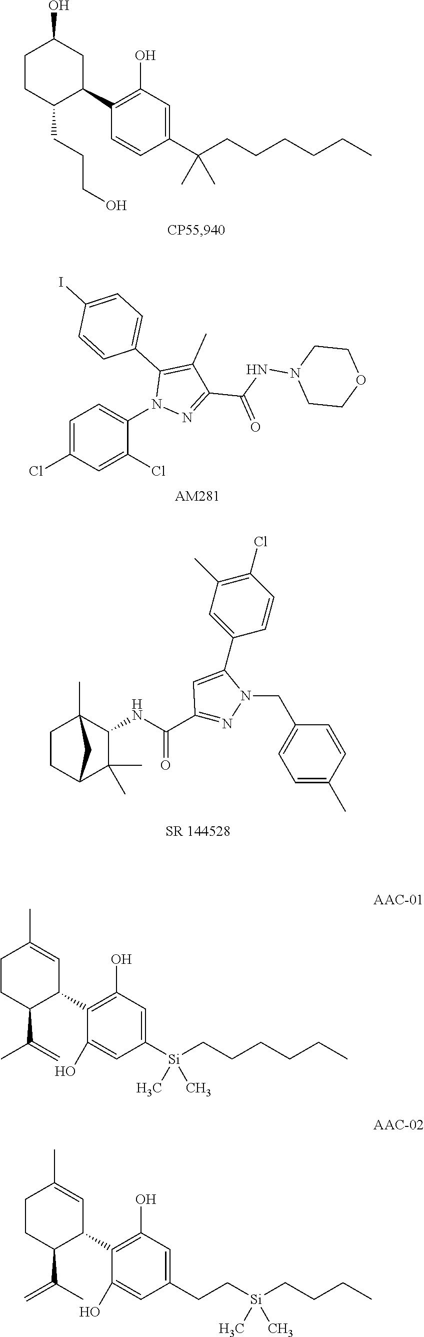

### CROSS-REFERENCES TO RELATED APPLICATIONS

The present application claims priority to U.S. Provisional Application No. 63/302,723 filed Jan. 25, 2022, the full disclosure of which is incorporated by reference in its entirety for all purposes.

### BACKGROUND

Neurodegenerative disorders (NDDs) are debilitating conditions resulting from the progressive degeneration of brain and central nervous system (CNS) function. As an example, epilepsy is one of the most common neurological disorders, affecting over 50 million patients of all ages worldwide. Epilepsy is a chronic brain disorder marked by sudden and recurrent sensory disturbance, loss of consciousness, and convulsions The global market for addressing epilepsy is anticipated to scale at a valuation of $9.5 billion by 2023, boosting growth for therapeutic treatments. Overall, neurodegenerative disease treatments have a $35 billion global market value, which is projected to grow to $62.8 billion by 2026. Further, the number of people affected by NDDs (including dementia, Alzheimer's disease (AD), and Parkinson's disease (PD)) is rapidly increasing as a consequence of an aging population.

Common molecular mechanisms of NDDs (i.e., inflammation, excitotoxicity, and mitochondrial dysfunction) have confirmed indicators that can be targeted therapeutically. A particular focus in NDD treatment is the endocannabinoid system, including cannabinoid receptors (CB₁ and CB₂), which has been shown to participate in neuroprotective mechanisms in several human and animal studies. Phytocannabinoids derived from Cannabis sativa, including cannabidiol (CBD) and isomers of tetrahydrocannabinol (Δ⁸- and Δ⁹-THC) (FIG. 1), have emerged as a powerful therapeutic class for treating neurological disorders and having promising potential as antipsychotics with neuroprotective properties. There is significant evidence supporting the benefits of CBD as a neuroprotective, antiepileptic, anxiolytic, antipsychotic, anti-inflammatory and anti-asthmatic agent, and also as an antagonist of tetrahydrocannabinol (Δ⁹-THC) psychoactive effects. Studies in transgenic animal models for AD and PD demonstrate the ability of cannabinoids to reduce neuroinflammation response and promote neurogenesis, acting as a preventative treatment for these disorders. In 2018, Epidiolex (CBD) became the first FDA-approved cannabinoid for the treatment of seizures associated with Lennox-Gastaut syndrome (LGS) and Dravet Syndrome (DS).

There are at least two significant problems that must be overcome to truly exploit the power of CBDs for the treatment of NDDs. First, there is a need for selective activation of the CB₂ receptor. This selective activation can provide an important strategy for the treatment of neurological disorders and inflammation while avoiding the psychoactive effects of CB₁ activation. Drugs acting selectively on the CB₂ receptor are therefore promising leads to combat CNS diseases, but the high degree of structural similarity in the architecture and binding pockets between agonist bound CB₂ and CB₁ imposes a substantial challenge in the design of a highly selective and differentiated CB₁/CB₂ agonists. Second, there is a need for more potent and bioavailable cannabinoid therapeutics. Pharmacokinetic data shows dose-dependent limitations of CBD in both plasma and brain concentrations with poor bioavailability (6%) from oral dosing. Thus, high doses of CBD are often required. Limitations due to effective drug delivery, cytotoxicity, neurotoxicity, and patient compliance further demonstrate the need for more potent and bioavailable cannabinoid therapeutics.

Among the available targets in the development of new therapeutics are the hydrophobic groups present in the chemical structure of CBD and other phytocannabinoids. There is a growing understanding that hydrophobic groups can be considered as pharmacophores and can be a significant element in the design of molecular probes and medicinal compounds, where it has been demonstrated that even the hydrophobic interaction of a protein residue and a single methyl group can stabilize binding as much as polar interactions (C. S. Leung, S. S. F. Leung, J. Tirado-Rives & W. L. Jorgensen, J. Med. Chem. 55, (2012): 4489). As such, it is important that the design and synthesis of compounds for biomedical research can efficiently modulate hydrophobic groups in addition to polar components such as heterocycles. In particular, there is a need for access to novel lipid analogs/derivatives with structural and conformational control. Design and control of hydrophobic substructures, including elements beyond carbon, can accomplish this goal while also expanding chemical space (D. Kajita et al., Bioorg. Med. Chem. Lett. 25, (2015): 3350). There are numerous examples of medicinal/bioactive compounds with lipid components (>4 carbon chains) important for their biological activity, such as Fingolimod as an anti-inflammatory and MS treatment (S. R. Shaikh, J. Nutr. Biochem. 23, (2012): 101; P. S. Espinosa & J. R. Berger, Mult. Scler. J. 17, (2011): 1387), cannabichromene as an anti-inflammatory and antibacterial (G. Wang et al., Bioorg. Chem. 72, (2017): 64), anandamide as a fatty acid neurotransmitter (H. de Morais et al., Eur. Neuropsychopharmacol. 26, (2016): 1590), 5-FU-C12 as an anti-cancer lipophilic analog of 5-fluorouracil (G. Lollo et al., J. Drug Target 0, (2018): 1), VCE-003.2 as a neuroprotective and anti-inflammatory (J. Diaz-Alonso et al., Sci. Rep. 6, (2016): 1), sphingosine-1-phosphate as a lipid mediator (P. S. Espinosa & J. R. Berger, Mult. Scler. J. 17, (2011): 1387), idebenone as a CoQio analog (D. M. Fash et al., Bioorg. Med. Chem. 21, (2013): 2346; A. Tomilov, S. Allen, C. K. Hui, A. Bettaieb & G. Cortopassi, Pharmacol. Res. 137, (2018): 89)), AUDA as an sEH inhibitor (I.-H. Kim et al., Bioorg. Med. Chem. 15, (2007): 312; J. Xu et al. Insect Biochem. Mol. Biol. 76, (2016): 62), and numerous heterocycle-lipid hybrid molecules that exhibit various bioactivity (V. Venepally & R. C. Reddy Jala, Eur. J. Med. Chem. 141, (2017): 113). There are also specific examples where chain length directly impacts biological activity. For example, the tetracaine anesthetic (A. Yamashita et al., Anesth. Analg. 97, (2003): 512), a cyclic nucleotide-gated (CNG) channel antagonist, exhibits a 5-fold increase with an octyl tail (N. A. Castle, J. Pharmacol. Exp. Ther. 255, (1990): 1038), while other reports have shown that longer lipophilic chains increase activity against HCT cancer cells (K. Bouhadir et al., Org. Commun. 10, (2017): 259), can affect inhibition of transient outward K+ current (N. A. Castle, J. Pharmacol. Exp. Ther. 255, (1990): 1038), and can alter agonism assay data for quorum-sensing N-acyl L-homoserine lactones (AHLs) (G. D. Geske et al., J. Am. Chem. Soc. 129, (2007): 13613; G. D. Geske et al., ChemBioChem 9, (2008): 389; J. P. Gerdt et al., ACS Chem. Biol. 12, (2017): 2457).

Silicon is one element useful as a carbon alternative for affecting the design and control of hydrophobic chemical structures. Based on its stability and unique properties, silicon can play an important role in the design of biological probes, pharmaceutical agents, and materials. Silicon is the 2nd most common element on earth, and silanes already have numerous industrial and technology applications in materials and inorganic chemistry. The flexible steric and substitution patterns of silyl groups allow tunable reactivity, stability, and solubility. There is no inherent “element-specific” toxicity or silicon-containing compounds, and silicon has multiple properties relevant for medicinal and clinical applications (G. A. Showell & J. S. Mills, Drug Discov. Today 8, (2003): 551; R. Ramesh & D. S. Reddy, J. Med. Chem. 61, (2018): 3779; W. Baines & R. Tacke, Curr. Opin. Drug Discov. Devel. 6, (2003): 526). The C—Si bond is stable under physiological conditions. Organosilanes can be synthesized using efficient reactions from a variety of commercially available reagents. The silicon atom has a larger covalent radius resulting in the formation of 20% longer Si—X bonds compared to C—X bonds and providing higher conformational flexibility (M. A. Brooks, Silicon in Organic, Organometallic, and Polymer Chemistry, Wiley: New York (2000)). The electropositive nature and bond-polarization of silicon (relative to C, N, and O) contributes to an electron-deficient center in a molecule. Trialkylsilyl groups are more lipophilic than the corresponding trialkylmethyl groups (Log P for trimethylsilyl-benzene=4.7 vs Log P for t-butylbenzene=4.0) (M. A. Brooks, Silicon in Organic, Organometallic, and Polymer Chemistry, Wiley: New York (2000); U. I. Zakai, G. Bikzhanova, D. Staveness, S. Gately & R. West, Appl. Organomet. Chem. 24, (2010): 189). And silicon has the potential to alter metabolic fate to avoid toxic metabolites (T. Johansson, L. Weidolf, F. Popp, R. Tacke & U. Jurva, Drug Metab. Dispos 38, (2010): 73; R. J. Fessednen & R. A. Hartman, J. Med. Chem. 13, (1970): 52; N. A. Meanwell, J. Med. Chem. 54, (1970): 2259).

The incorporation of silicon isosteres into known drug scaffolds is a method to optimize biological activity and reduce toxicity (G. A. Showell & J. S. Mills, Drug Discov. Today 8, (2003): 551; S. Gately & R. West, Drug Dev. Res. 68, (2007): 156; M. Geyer et al., ChemMedChem 10, (2015): 911; A. K. Franz & S. O. Wilson, J. Med. Chem. 56, (2013): 388). Silatecans, silyl analogs of DNA topoisomerase inhibitor camptothecin, have been shown to increase cell penetration, enhance blood stability and improve pharmacokinetics relative to camptothecin (A. A. Gabizon et al., Clin. Cancer Res. 12, (2006): 1913). The enhanced in vivo stability of DB-67 is attributed to the enhanced lipophilicity that reduces in vivo hydrolysis due to increased partitioning in the red blood cells (V. J. Venditto & E. E. Simanek, Mol. Pharm. 7, (2010): 307). Notably, silatecans Karenitecin and DB-67 have advanced to clinical trials (A. A. Gabizon et al., Clin. Cancer Res. 12, (2006): 1913; A. Daud et al., Clin. Cancer Res. 11, (2005): 3009). Several trimetylsilylpyrazole analogs of BIRB-796, a non-peptide inhibitor of p38 mitogen-activated protein (MAP) kinase (J. Regan et al., J. Med. Chem. 45, (2002): 2994; W. N. Sivak et al., Acta Biomater. 4, (2008): 852), have been synthesized as new silicon isosteres for kinase inhibitors (V. J. Venditto & E. E. Simanek, Mol. Pharm. 7, (2010): 307). A sila-analog of BIRB-796 demonstrated enhanced stability to degradation by human liver microsomes and in-vivo data in an LPS-induced model of TNF-α release indicated similar efficacy and also suggested that the silicon analog induces TNF-α suppression more quickly (59% compared to 41% at 30 min) (J. Regan et al., J. Med. Chem. 45, (2002): 2994). Incorporating a silicon-containing amino acid such as γ-(dimethylsila) proline (silaproline, Sip) (M. W. Mutahi, T. Nittoli, L. Guo, & S. M. N. Sieburth, J. Am. Chem. Soc. 124, (2002): 7363; S. Pujals et al., J. Am. Chem. Soc. 128, (2006): 8479), has been shown to increase resistance to proteolytic degradation, increase cellular uptake, and enhance lipophilicity (e.g. Log P is 0.094 for Fmoc-Pro and 1.3 for Fmoc-Sip) (S. Pujals et al., J. Am. Chem. Soc. 128, (2006): 8479). Upon incorporation of silaproline in proline-rich cell-penetrating peptides, the peptides maintained their secondary structure and cellular uptake was enhanced (F. Cavelier et al., J. Am. Chem. Soc. 124, (2002): 2917; F. Cavelier, D. Marchand, J. Martinez & S. Sagan, J. Pepet. Res. 63, (2004): 290). TMS-alanine has been used as a replacement for both phenylalanine and for leucine based on the lipophilicity (R. Fanelli et al., J. Med. Chem. 58, (2015): 7785).

The metabolism of organosilicon molecules is an important consideration where similar metabolic rates and oxidation occurs (R. J. Fessednen &R. A. Hartman, J. Med. Chem. 13, (1970): 52; M. Geyer et al., ChemMedChem 10, (2015): 911), however, the presence of the silicon atom can also alter the metabolic fate to reduce toxicity. For example, sila-haloperidol, a dopamine D2 antagonist, avoids formation of a neurotoxic pyridinium ion metabolite (T. Johansson, L. Weidolf, F. Popp, R. Tacke & U. Jurva, Drug Metab. Dispos 38, (2010): 73; B. Subramanyam, H. Rollema, T. Woolf & N. Castagnoli, Biochem. Biophys. Res. Commun. 16, (1990): 238; R. Tacke et al., ChemMedChem 3, (2008): 152). In general, organosilicon molecules are stable in aqueous and oxygen-rich environments and the metabolism of organosilanes is still generally shown to follow standard pathways.

Despite these examples, there has not been a systematic study for incorporation of silyl groups to strategically alter the structure and conformation of hydrophobic groups for medicinal chemistry. In particular, the inventors are aware of very few reports describing silyl groups in incorporated in a fatty acid (D. Kajita et al., Bioorg. Med. Chem. Lett. 25, (2015): 3350; U.S. Pat. No. 8,895,769). In view of these limitations in the available knowledge related to silyl lipids, as well as the aforementioned shortcomings in applying existing cannabinoid analogs for treating neurodegenerative and inflammation-related disorders, there are currently unmet needs for additional approaches that can provide improved compounds with enhanced therapeutic efficacy and selectivity. The materials and methods disclosed herein address these and other needs.

### BRIEF SUMMARY

The present disclosure generally relates to the design, synthesis, and use of silyl-lipid cannabinoids as innovative carbon-silicon bioisosteres having agonist activity towards CB_{1/2} receptors. In particular, biological testing disclosed herein shows selective and potent agonism of the CB₂ receptor with the provided silyl-lipid structures. The silyl-containing cannabinoids (based on, e.g., CBD, Δ⁸-THC and Δ⁹-THC) have lipid structures representing an important new chemical space for biomedical and cannabis research for neurodegenerative disorders.

In one aspect, the disclosure is to a compound having the structure of formula I:

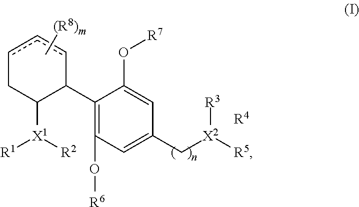

or a pharmaceutically acceptable salt, solvate, or isomer thereof. R¹ and R² of formula I may each independently be hydrogen, C₁₋₆ alkyl, ═CH₂, or C₂₋₆ alkenyl. R³ and R⁴ of formula I may each independently be hydrogen, C₁₋₆ alkyl, ═CH₂, or C₂₋₆ alkenyl, or may be combined with X² to form a 3- to 12-membered heterocyclyl. R⁵ of formula I may be hydrogen, C₁₋₉ alkyl, ═CH₂, C₂₋₆ alkenyl, C₆₋₁₂ aryl, or C₁₋₆ alkyl-C₆₋₁₂ aryl, where the aryl and the alkyl-aryl are optionally substituted with 1-4 R⁹ groups. R⁶ of formula I may be hydrogen, C₁₋₆ alkyl, or a bond connected to X¹. R⁷ of formula I may be hydrogen or C₁₋₆ alkyl. Each R⁸ and R⁹ of formula I may independently be C₁₋₆ alkyl, hydroxy, C₁₋₆ alkoxy, C₁₋₆ hydroxyalkyl, halogen, C₁₋₆ haloalkyl, nitro, or cyano. X¹ and X² of formula I may each independently be C or Si, with the proviso that at least one of X¹ and X² is Si. Subscript m of formula I may be an integer from 0 to 4. Subscript n of formula I may be an integer from 0 to 9.

In another aspect, the disclosure is to a pharmaceutical composition. The pharmaceutical composition includes any of the compounds as disclosed herein. The pharmaceutical composition further includes a pharmaceutically acceptable carrier.

In another aspect, the disclosure is to a method of preparing a compound having the structure:

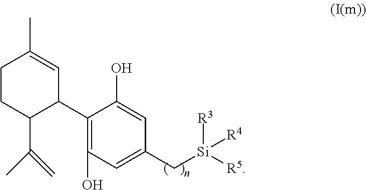

The method includes forming a silyl cannabinoid synthesis reaction mixture comprising 1-methyl-4-(prop-1-en-2-yl)cyclohex-2-en-1-ol and a compound of formula II:

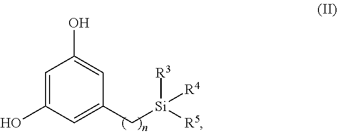

Under conditions sufficient to form the compound of formula I(m). R³ and R⁴ of formulas I(m) and II may each independently be hydrogen, C₁₋₆ alkyl, ═CH₂, or C₂₋₆ alkenyl, or may be combined with X² to form a 3- to 12-membered heterocyclyl. R⁵ of formulas I(m) and II may be hydrogen, C₁₋₉ alkyl, ═CH₂, C₂₋₆ alkenyl, C₆₋₁₂ aryl, or C₁₋₆ alkyl-C₆₋₁₂ aryl, where the aryl and the alkyl-aryl are optionally substituted with 1-4 R⁹ groups. Each R⁹ may independently be C₁₋₆ alkyl, hydroxy, C₁₋₆ alkoxy, C₁₋₆ hydroxyalkyl, halogen, C₁₋₆ haloalkyl, nitro, or cyano. Subscript n of formulas I(m) and II may be an integer from 0 to 9.

In another aspect, the disclosure is to a method of treating a disorder. The method includes administering to a subject in need thereof a therapeutically effective amount of any of the compounds disclosed herein or any of the pharmaceutical compositions disclosed herein.

### BRIEF DESCRIPTION OF THE DRAWINGS

FIG. 1 presents the chemical structures of exemplary cannabinoid compounds.

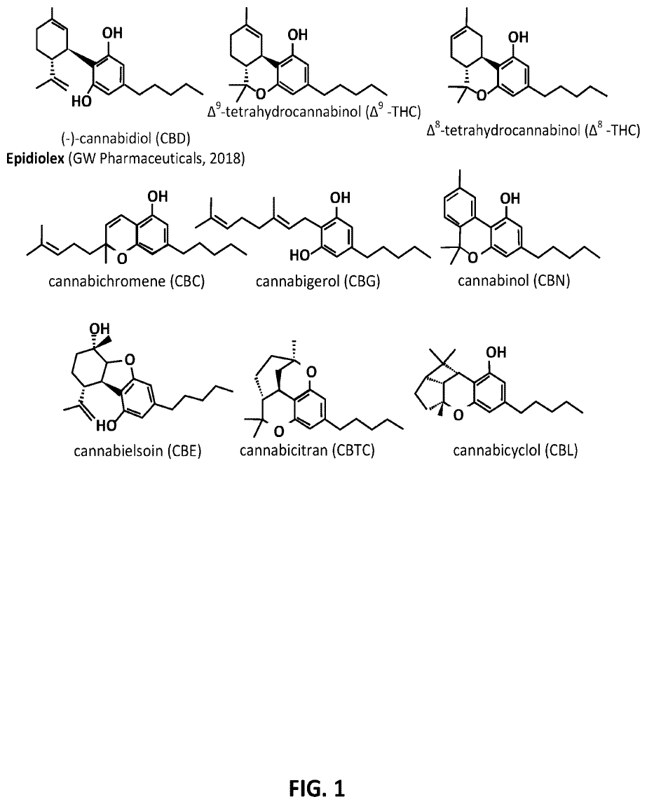

FIG. 2 presents the chemical structures of exemplary silane building blocks suitable for use with the provided synthetic methods for producing the provided silyl lipid cannabinoid compounds.

FIG. 3 illustrates an exemplary reaction scheme in accordance with a provided embodiment.

FIG. 4 illustrates exemplary reaction schemes for producing silyl lipid cannabinoid compounds in accordance with a provided embodiment.

FIG. 5 illustrates an exemplary reaction scheme in accordance with a provided embodiment.

FIG. 6 illustrates exemplary reaction schemes in accordance with a provided embodiment.

FIG. 7 presents the chemical structures of control compounds and silyl lipid cannabinoid compounds in accordance with a provided embodiment.

### DETAILED DESCRIPTION

### I. General

The present disclosure provides compounds capable of exhibiting agonist activity towards one or both of cannabinoid receptors CB₂ and CB₁. Beneficially, this activity of the provided compounds can be highly potent and selective, especially when compared to the potency and selectivity of other known cannabinoids and cannabinoid analogs. The advantageous properties of the compounds disclosed herein render them particularly suitable for use in effective therapies targeting treatment of disorders involving neurodegeneration and/or inflammation. For example, the provided compounds can be useful in methods for treating epilepsy.

The disclosure demonstrates synthesis of a collection of silyl-lipid analogs of CBD, as well as profiling of the CB₁ and CB₂ agonism activity for these silyl-CBD compounds. In some embodiments, the provided compounds include strategic incorporation of a silyldimethyl group in the alkyl chain of CBD analogs to adjust, e.g., chain-length, conformation, and branching, providing access to unique silyl-lipid derivatives of cannabinoids having advantageous properties. The provided silicon-carbon bioisosteres may have improved PK/PD (pharmacodynamic) properties. For example, silicon incorporation may increase brain to plasma ratio, due to the increased lipophilicity and improved plasma and microsomal stability of the provided compounds. Notably, there is no inherent “element-specific” toxicity of silicon-containing compounds and the Si—C bond is also stable under physiological conditions. The presence of silicon may also alter metabolic pathways in some cases, further reducing toxicity.

The provided compounds may also promote selective activation of the CB₂ receptor and modulate and improve PK/ADME (absorption, distribution, metabolism, elimination) properties compared to analogs of known cannabinoids. Particularly, the position/location of the silyl group may control CB₂-selective activity. While the provided modular synthetic approach can target the silicon-bioisostere of the 1,1-dimethylheptyl alkyl-side chain, the approach may also be applied to access various silyl-lipid tails with aryl/alkyl modifications or chain length modifications. Such modification of the THC/CBD alkyl chain of the provided compounds may also increase potency and selectivity. For example, CBD analogs with longer, bulkier, and more hydrophobic alkyl side chains may exhibit improved therapeutic properties. For example, long alkyl chains are able to extend into a long hydrophobic tunnel of CB₁ maximizing hydrophobic interactions with the residues of the channel.

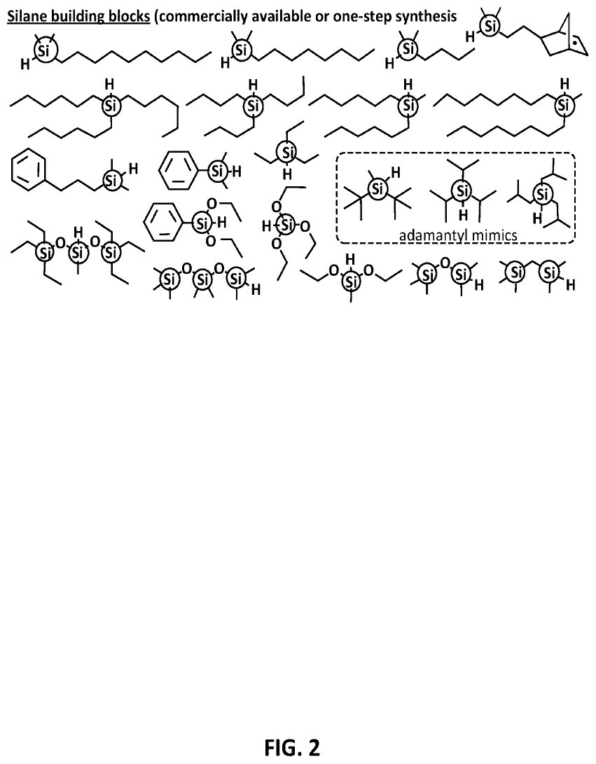

The provided synthetic methods can access a series of silicon-containing resorcinyl derivatives from readily available starting materials (FIG. 2) through nucleophilic substitution and hydrosilylation. Using the provided synthetic approaches, the length of the lipid and position of the dimethylsilyl group along the lipid tail can be easily modified. The synthetic strategy also offers the opportunity to synthesize and evaluate tri-alkyl silane and dimethylaryl-silane analogs for which the carbon counterparts are readily accessible. In addition to producing silyl-CBD analogs, the provided routes can also be applied to synthesize silyl-THC analogs. Silyl-THC analogs (Δ⁸ and Δ⁹ isomers) can be accessed, for example, from p-methadienol with silyl-resorcinyl derivatives through cyclization of CBD. Based on commercially available silanes, a collection of numerous structures of silyl-CBD, silyl-THC, silyl-CBN and silyl-CBC molecules, including analogs of rare and often understudied cannabinoids, such as cannabinol (CBN) and cannabichromene (CBC), can be readily generated. The approach also allows access of the unnatural enantiomer for each compound.

### II. Definitions

The abbreviations used herein have their conventional meaning within the chemical and biological arts. Description of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, or physiological conditions. Where substituent groups are specified by their conventional chemical formulae, written from left to right, they equally encompass the chemically identical substituents that would result from writing the structure from right to left, e.g., —CH₂O— is equivalent to —OCH₂—.

As used herein, the term “salt” refers to acid or base salts of the compounds used in the methods of the present disclosure. A “pharmaceutically acceptable salt” is one that is compatible with other ingredients of a formulation composition containing the compound, and that is not deleterious to a recipient thereof. It is thus understood that the pharmaceutically acceptable salts are non-toxic. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds of the present disclosure are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

As used herein, the term “solvate” refers to a compound that is complexed to at least one solvent molecule. The compounds of the present disclosure may be complexed with from 1 to 10 solvent molecules. In some embodiments, the solvent is water and the solvate is a hydrate.

As used herein, the term “isomers” refers to compounds having the same number and kind of atoms, and hence the same molecular weight, but differing in respect to the structural arrangement or configuration of the atoms. Isomers thus include compounds having different arrangements of the same formula of atoms in a molecule possessing one or more asymmetric carbon atoms or double bonds. Isomers may include racemates, enantiomers, diastereomers, geometric isomers, and individual isomers.

As used herein, the term “alkyl” refers to a straight or branched, saturated, aliphatic radical having the number of carbon atoms indicated. Alkyl may include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆, and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl may also refer to alkyl groups having up to 20 carbons atoms, such as, but not limited to heptyl, octyl, nonyl, decyl, etc.

As used herein, the term “alkenyl” refers to a straight chain or branched hydrocarbon having at least 2 carbon atoms and at least one double bond. Alkenyl may include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups may have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, and 1,3,5-hexatrienyl. Alkenyl groups can be substituted or unsubstituted.

As used herein, the term “alkoxy” refers to a substituted alkyl group, as defined above, having an oxygen atom that connects the alkyl group to the point of attachment: alkyl-O—. As for the (unsubstituted) alkyl group, alkoxy groups may have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc.

As used herein, the term “hydroxy” refers to a group consisting of hydrogen bonded to oxygen: —OH.

As used herein, the term “hydroxyalkyl” refers to a substituted alkyl group, as defined above, where at least one of the hydrogen atoms is replaced with a hydroxy group. As for the (unsubstituted) alkyl group, hydroxyalkyl groups may have any suitable number of carbon atoms, such as C₁₋₆. Exemplary hydroxyaryl groups include, but are not limited to, hydroxymethyl, hydroxyethyl (where the hydroxy is in the 1- or 2-position), hydroxypropyl (where the hydroxy is in the 1-, 2- or 3-position), hydroxybutyl (where the hydroxy is in the 1-, 2-, 3- or 4-position), hydroxypentyl (where the hydroxy is in the 1-, 2-, 3-, 4- or 5-position), hydroxyhexyl (where the hydroxy is in the 1-, 2-, 3-, 4-, 5- or 6-position), 1,2-dihydroxyethyl, and the like.

As used herein, the term “aryl” refers to an aromatic ring system having any suitable number of ring atoms and any suitable number of rings. Aryl groups may include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups may be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups, such as phenyl, naphthyl and biphenyl, have from 6 to 12 ring members.

As used herein, the term “alkyl-aryl” refers to a radical having an alkyl component and an aryl component, each as defined above, where the alkyl component links the aryl component to the point of attachment. The alkyl component is as defined above, except that the alkyl component is at least divalent, i.e., is an alkylene, to link to the aryl component and to the point of attachment. The alkyl component can include any number of carbons, such as C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆, and C₅₋₆. Examples of the alkyl-alkoxy group include, but are not limited to, 2-ethoxy-ethyl and methoxymethyl. Exemplary alkyl-aryl groups include, but are not limited to, phenylmethyl, phenylethyl (where the phenyl is in the 1- or 2-position), phenylpropyl (where the phenyl is in the 1-, 2- or 3-position), phenylbutyl (where the phenyl is in the 1-, 2-, 3- or 4-position), phenylpentyl (where the phenyl is in the 1-, 2-, 3-, 4- or 5-position), phenylhexyl (where the phenyl is in the 1-, 2-, 3-, 4-, 5- or 6-position), 1,2-diphenylethyl, and the like.

As used herein, the term “heterocyclyl” refers to a saturated or partially unsaturated non-aromatic ring or a partially non-aromatic multiple-ring system in which one or more of the carbon atoms are each independently replaced with the same or different heteroatom such as N, O, or S. Additional heteroatoms can also be useful, including, but not limited to, B, Al, Si and P. The heteroatoms can also be oxidized, such as, but not limited to, —S(O)— and —S(O)₂—. Heterocycles include, but are not limited to, groups derived from azetidine, aziridine, imidazolidine, morpholine, oxirane (epoxide), oxetane, piperazine, piperidine, pyrazolidine, piperidine, pyrrolidine, pyrrolidinone, tetrahydrofuran, tetrahydrothiophene, dihydropyridine, tetrahydropyridine, tetrahydro-2H-thiopyran 1,1-dioxide, quinuclidine, N-bromopyrrolidine, N-chloropiperidine, and the like. Heterocyclyl groups also include partially unsaturated ring systems containing one or more double bonds, including fused ring systems with one aromatic ring and one non-aromatic ring, but not fully aromatic ring systems. Examples include dihydroquinolines, e.g., 3,4-dihydroquinoline, dihydroisoquinolines, e.g., 1,2-dihydroisoquinoline, dihydroimidazole, tetrahydroimidazole, etc., indoline, isoindoline, isoindolones (e.g., isoindolin-1-one), isatin, dihydrophthalazine, quinolinone, spiro[cyclopropane-1,1′-isoindolin]-3′-one, and the like. Heterocyclyl groups may have 3-12 members, or 3-10 members, or 3-7 members, or 5-6 members.

As used herein, the term “halogen” refers to fluorine, chlorine, bromine, and iodine.

As used herein, the term “haloalkyl” refers to a substituted alkyl, as defined above, where at least one of the hydrogen atoms is replaced with a halogen atom. As for the (unsubstituted) alkyl group, haloalkyl groups may have any suitable number of carbon atoms, such as C₁₋₆. For example, haloalkyl includes trifluoromethyl, fluoromethyl, etc. In some instances, the term “perfluoro” may be used to define a compound or radical where all the hydrogens are replaced with fluorine. For example, perfluoromethane includes 1,1,1-trifluoromethyl.

As used herein, the term “nitro” refers to a group consisting of two oxygen atoms bonded to nitrogen: —NO₂.

As used herein, the term “cyano” refers to a group consisting of nitrogen triple-bonded to carbon: —C═N.

As used herein, the term “silyl” refers to a group having at least one carbon atom (alkyl groups) attached to Si: —Si—C.

As used herein, the term “siloxy” refers to a group having a silicon atom bonded to oxygen: —Si—O.

As used herein, the term “agonist” refers to a substance that has an affinity for the active site of a receptor and thereby preferentially stabilizes the active state of the receptor, or a substance that produces activation of receptors and enhances signaling by those receptors.

As used herein, the term “composition” refers to a product comprising the specified ingredients in the specified amounts, as well as any product, which results, directly or indirectly, from combination of the specified ingredients in the specified amounts. A “pharmaceutically acceptable composition” is one in which each ingredients, e.g., a carrier, diluent or excipient, is compatible with the other ingredients of a formulation composition and not deleterious to the recipient thereof.

As used herein, the terms “pharmaceutically acceptable carrier” and “pharmaceutically acceptable excipient” refer to a substance that aids the administration of an active agent to and absorption by a subject and may be included in the compositions of the present disclosure without causing a significant adverse toxicological effect on the subject. Non-limiting examples of pharmaceutically acceptable excipients and carriers include water, NaCl, normal saline solutions, normal sucrose, normal glucose, binders, fillers, disintegrants, lubricants, coatings, and the like. One of skill in the art will recognize that other pharmaceutically acceptable excipients and carriers are useful in the present disclosure.

As used herein, the terms “treat,” “treating,” and “treatment” refer to a procedure resulting in any indicia of success in the elimination or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of one or more symptoms. The treatment or amelioration of symptoms can be based on any objective or subjective parameter; including, e.g., the result of a physical examination or laboratory test.

As used herein, the term “administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

As used herein, the term “subject” refers to a vertebrate, and preferably to a mammal. Mammalian subjects for which the provided composition is suitable include, but are not limited to, mice, rats, simians, humans, farm animals, sport animals, and pets. In some embodiments, the subject is human. In some embodiments, the subject is male. In some embodiments, the subject is female. In some embodiments, the subject is an adult. In some embodiments, the subject is an adolescent. In some embodiments, the subject is a child. In some embodiments, the subject is above 10 years of age, e.g., above 20 years of age, above 30 years of age, above 40 years of age, above 50 years of age, above 60 years of age, above 70 years of age, or above 80 years of age. In some embodiments, the subject is less than 80 years of age, e.g., less than 70 years of age, less than 60 years of age, less than 50 years of age, less than 40 years of age, less than 30 years of age, less than 20 years of age, or less than 10 years of age.

As used herein, the term “therapeutically effective amount” refers to an amount or dose of a compound, composition, or formulation that produces therapeutic effects for which it is administered. The exact amount or dose will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the terms “including,” “comprising,” “having,” “containing,” and variations thereof, are inclusive and open-ended and do not exclude additional, unrecited elements or method steps beyond those explicitly recited. As used herein, the phrase “consisting of” is closed and excludes any element, step, or ingredient not explicitly specified. As used herein, the phrase “consisting essentially of” limits the scope of the described feature to the specified materials or steps and those that do not materially affect the basic and novel characteristics of the disclosed feature.

As used herein, the singular forms “a”, “an” and “the” include plural referents unless the content clearly dictates otherwise. Thus, for example, reference to “a pharmaceutically acceptable carrier” optionally includes a combination of two or more pharmaceutically acceptable carriers, and the like.

### III. Compounds

The present disclosure provides many silyl-lipid cannabinoid analogs. In some embodiments, the disclosure provides compounds having the structure of formula I:

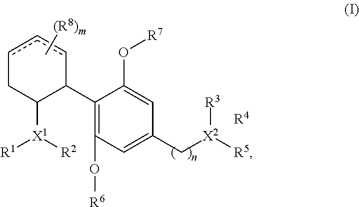

where R¹ and R² of formula I can independently be hydrogen, C₁₋₆ alkyl, ═CH₂, or C₂₋₆ alkenyl. R³ and R⁴ of formula I can independently be hydrogen, C₁₋₆ alkyl, ═CH₂, or C₂₋₆ alkenyl, or can be combined with X² to form a 3- to 12-membered heterocyclyl. R⁵ of formula I can be hydrogen, C₁₋₉ alkyl, ═CH₂, C₂₋₆ alkenyl, C₆₋₁₂ aryl, or C₁₋₆ alkyl-C₆₋₁₂ aryl, where the aryl and the alkyl-aryl are optionally substituted with 1-4 R⁹ groups. R⁶ of formula I can be hydrogen, C₁₋₆ alkyl, or a bond connected to X¹. R⁷ of formula I is hydrogen or C₁₋₆ alkyl. Each R⁸ and R⁹ can independently be C₁₋₆ alkyl, hydroxy, C₁₋₆ alkoxy, C₁₋₆ hydroxyalkyl, halogen, C₁₋₆ haloalkyl, nitro, or cyano. X¹ and X² of formula I can each independently be C or Si, with the proviso that at least one of X¹ and X² is Si. Subscript m of formula I can be an integer from 0 to 4. Subscript n can be an integer from 0 to 9. The provided compounds include pharmaceutically acceptable salts, solvates, and isomers of compounds with the structure of formula I.

In some embodiments, R¹ of formula I is C₁₋₆ alkyl or ═CH₂. In some embodiments, R¹ is C₁₋₆ alkyl. In some embodiments, R¹ is methyl. In some embodiments, R¹ is ethyl. In some embodiments, R¹ is propyl. In some embodiments, R¹ is isopropyl. In some embodiments, R¹ is butyl. In some embodiments, R¹ is isobutyl. In some embodiments, R¹ is sec-butyl. In some embodiments, R¹ is tert-butyl. In some embodiments, R¹ is pentyl. In some embodiments, R¹ is isopentyl. In some embodiments, R¹ is 2-methylbutyl. In some embodiments, R¹ is pentan-2-yl. In some embodiments, R¹ is 3-methylbutan-2-yl. In some embodiments, R¹ is pentan-3-yl. In some embodiments, R¹ is neopentyl. In some embodiments, R¹ is tert-pentyl. In some embodiments, R¹ is hexyl. In some embodiments, R¹ is 4-methylpentyl. In some embodiments, R¹ is 3-methylpentyl. In some embodiments, R¹ is 2-methylpentyl. In some embodiments, R¹ is hexan-2-yl. In some embodiments, R¹ is 2,3-dimethylbutyl. In some embodiments, R¹ is 4-methylpentan-2-yl. In some embodiments, R¹ is 3-methylpentan-2-yl. In some embodiments, R¹ is 2-ethylbutyl. In some embodiments, R¹ is hexan-3-yl. In some embodiments, R¹ is 3,3-dimethylbutyl. In some embodiments, R¹ is 2,2-dimethylbutyl. In some embodiments, R¹ is 2-methylpentan-2-yl. In some embodiments, R¹ is ═CH₂. In some embodiments, R¹ is hydrogen. In some embodiments, R¹ is C₂₋₆ alkenyl.

In some embodiments, R² of formula I is C₁₋₆ alkyl or ═CH₂. In some embodiments, R² is C₁₋₆ alkyl. In some embodiments, R² is methyl. In some embodiments, R² is ethyl. In some embodiments, R² is propyl. In some embodiments, R² is isopropyl. In some embodiments, R² is butyl. In some embodiments, R² is isobutyl. In some embodiments, R² is sec-butyl. In some embodiments, R² is tert-butyl. In some embodiments, R² is pentyl. In some embodiments, R² is isopentyl. In some embodiments, R² is 2-methylbutyl. In some embodiments, R² is pentan-2-yl. In some embodiments, R² is 3-methylbutan-2-yl. In some embodiments, R² is pentan-3-yl. In some embodiments, R² is neopentyl. In some embodiments, R² is tert-pentyl. In some embodiments, R² is hexyl. In some embodiments, R² is 4-methylpentyl. In some embodiments, R¹ is 3-methylpentyl. In some embodiments, R¹ is 2-methylpentyl. In some embodiments, R² is hexan-2-yl. In some embodiments, R² is 2,3-dimethylbutyl. In some embodiments, R² is 4-methylpentan-2-yl. In some embodiments, R² is 3-methylpentan-2-yl. In some embodiments, R² is 2-ethylbutyl. In some embodiments, R² is hexan-3-yl. In some embodiments, R² is 3,3-dimethylbutyl. In some embodiments, R² is 2,2-dimethylbutyl. In some embodiments, R² is 2-methylpentan-2-yl. In some embodiments, R² is-CH₂. In some embodiments, R² is hydrogen. In some embodiments, R² is C₂₋₆ alkenyl.

In some embodiments, R³ of formula I is C₁₋₆ alkyl. In some embodiments, R³ is methyl. In some embodiments, R³ is ethyl. In some embodiments, R³ is propyl. In some embodiments, R³ is isopropyl. In some embodiments, R³ is butyl. In some embodiments, R³ is isobutyl. In some embodiments, R³ is sec-butyl. In some embodiments, R³ is tert-butyl. In some embodiments, R³ is pentyl. In some embodiments, R³ is isopentyl. In some embodiments, R³ is 2-methylbutyl. In some embodiments, R³ is pentan-2-yl. In some embodiments, R³ is 3-methylbutan-2-yl. In some embodiments, R³ is pentan-3-yl. In some embodiments, R³ is neopentyl. In some embodiments, R³ is tert-pentyl. In some embodiments, R³ is hexyl. In some embodiments, R³ is 4-methylpentyl. In some embodiments, R³ is 3-methylpentyl. In some embodiments, R³ is 2-methylpentyl. In some embodiments, R³ is hexan-2-yl. In some embodiments, R³ is 2,3-dimethylbutyl. In some embodiments, R³ is 4-methylpentan-2-yl. In some embodiments, R³ is 3-methylpentan-2-yl. In some embodiments, R³ is 2-ethylbutyl. In some embodiments, R³ is hexan-3-yl. In some embodiments, R³ is 3,3-dimethylbutyl. In some embodiments, R³ is 2,2-dimethylbutyl. In some embodiments, R³ is 2-methylpentan-2-yl. In some embodiments, R³ is hydrogen. In some embodiments, R³ is-CH₂. In some embodiments, R³ is C₂₋₆ alkenyl.

In some embodiments, R⁴ of formula I is C₁₋₆ alkyl. In some embodiments, R⁴ is methyl. In some embodiments, R⁴ is ethyl. In some embodiments, R⁴ is propyl. In some embodiments, R⁴ is isopropyl. In some embodiments, R⁴ is butyl. In some embodiments, R⁴ is isobutyl. In some embodiments, R⁴ is sec-butyl. In some embodiments, R⁴ is tert-butyl. In some embodiments, R⁴ is pentyl. In some embodiments, R⁴ is isopentyl. In some embodiments, R⁴ is 2-methylbutyl. In some embodiments, R⁴ is pentan-2-yl. In some embodiments, R⁴ is 3-methylbutan-2-yl. In some embodiments, R⁴ is pentan-3-yl. In some embodiments, R⁴ is neopentyl. In some embodiments, R⁴ is tert-pentyl. In some embodiments, R⁴ is hexyl. In some embodiments, R⁴ is 4-methylpentyl. In some embodiments, R⁴ is 3-methylpentyl. In some embodiments, R⁴ is 2-methylpentyl. In some embodiments, R⁴ is hexan-2-yl. In some embodiments, R⁴ is 2,3-dimethylbutyl. In some embodiments, R⁴ is 4-methylpentan-2-yl. In some embodiments, R⁴ is 3-methylpentan-2-yl. In some embodiments, R⁴ is 2-ethylbutyl. In some embodiments, R⁴ is hexan-3-yl. In some embodiments, R⁴ is 3,3-dimethylbutyl. In some embodiments, R⁴ is 2,2-dimethylbutyl. In some embodiments, R⁴ is 2-methylpentan-2-yl. In some embodiments, R⁴ is hydrogen. In some embodiments, R⁴ is ═CH₂. In some embodiments, R⁴ is C₂₋₆ alkenyl.

In some embodiments, R⁵ of formula I is C₁₋₆ alkyl. In some embodiments, R⁵ is methyl. In some embodiments, R⁵ is ethyl. In some embodiments, R⁵ is propyl. In some embodiments, R⁵ is isopropyl. In some embodiments, R⁵ is butyl. In some embodiments, R⁵ is isobutyl. In some embodiments, R⁵ is sec-butyl. In some embodiments, R⁵ is tert-butyl. In some embodiments, R⁵ is pentyl. In some embodiments, R⁵ is isopentyl. In some embodiments, R⁵ is 2-methylbutyl. In some embodiments, R⁵ is pentan-2-yl. In some embodiments, R⁵ is 3-methylbutan-2-yl. In some embodiments, R⁵ is pentan-3-yl. In some embodiments, R⁵ is neopentyl. In some embodiments, R⁵ is tert-pentyl. In some embodiments, R⁵ is hexyl. In some embodiments, R⁵ is 4-methylpentyl. In some embodiments, R⁵ is 3-methylpentyl. In some embodiments, R⁵ is 2-methylpentyl. In some embodiments, R⁵ is hexan-2-yl. In some embodiments, R⁵ is 2,3-dimethylbutyl. In some embodiments, R⁵ is 4-methylpentan-2-yl. In some embodiments, R⁵ is 3-methylpentan-2-yl. In some embodiments, R⁵ is 2-ethylbutyl. In some embodiments, R⁵ is hexan-3-yl. In some embodiments, R⁵ is 3,3-dimethylbutyl. In some embodiments, R⁵ is 2,2-dimethylbutyl. In some embodiments, R⁵ is 2-methylpentan-2-yl. In some embodiments, R⁵ is any of the aforementioned C₁₋₆ alkyl groups substituted with 1 to 4 R⁹ groups.

In some embodiments, R⁵ of formula I is C₆₋₁₂ aryl. In some embodiments, R⁵ is C₆₋₁₂ phenyl. In some embodiments, R⁵ is naphthyl. In some embodiments, R⁵ is biphenyl. In some embodiments, R⁵ is acenaphthyl. In some embodiments, R⁵ is fluorenyl. In some embodiments, R⁵ is indenyl. In some embodiments, R⁵ is azulenyl. In some embodiments, R⁵ is hydrogen. In some embodiments, R⁵ is-CH₂. In some embodiments, R⁵ is C₂₋₆ alkenyl. In some embodiments, R⁵ is C₁₋₆ alkyl-C₆₋₁₂ aryl. In some embodiments, R⁵ is any of the aforementioned aryl, alkenyl, or alkyl-aryl groups substituted with 1 to 4 R⁹ groups.

In some embodiments, R⁶ of formula I is C₁₋₆ alkyl. In some embodiments, R⁶ is methyl. In some embodiments, R⁶ is ethyl. In some embodiments, R⁶ is propyl. In some embodiments, R⁶ is isopropyl. In some embodiments, R⁶ is butyl. In some embodiments, R⁶ is isobutyl. In some embodiments, R⁶ is sec-butyl. In some embodiments, R⁶ is tert-butyl. In some embodiments, R⁶ is pentyl. In some embodiments, R⁶ is isopentyl. In some embodiments, R⁶ is 2-methylbutyl. In some embodiments, R⁶ is pentan-2-yl. In some embodiments, R⁶ is 3-methylbutan-2-yl. In some embodiments, R⁶ is pentan-3-yl. In some embodiments, R⁶ is neopentyl. In some embodiments, R⁶ is tert-pentyl. In some embodiments, R⁶ is hexyl. In some embodiments, R⁶ is 4-methylpentyl. In some embodiments, R⁶ is 3-methylpentyl. In some embodiments, R⁶ is 2-methylpentyl. In some embodiments, R⁶ is hexan-2-yl. In some embodiments, R⁶ is 2,3-dimethylbutyl. In some embodiments, R⁶ is 4-methylpentan-2-yl. In some embodiments, R⁶ is 3-methylpentan-2-yl. In some embodiments, R⁶ is 2-ethylbutyl. In some embodiments, R⁶ is hexan-3-yl. In some embodiments, R⁶ is 3,3-dimethylbutyl. In some embodiments, R⁶ is 2,2-dimethylbutyl. In some embodiments, R⁶ is 2-methylpentan-2-yl. In some embodiments, R⁶ is a bond connected to X¹. In some embodiments, R⁶ is hydrogen.

In some embodiments, R⁷ of formula I is C₁₋₆ alkyl. In some embodiments, R⁷ is methyl. In some embodiments, R⁷ is ethyl. In some embodiments, R⁷ is propyl. In some embodiments, R⁷ is isopropyl. In some embodiments, R⁷ is butyl. In some embodiments, R⁷ is isobutyl. In some embodiments, R⁷ is sec-butyl. In some embodiments, R⁷ is tert-butyl. In some embodiments, R⁷ is pentyl. In some embodiments, R⁷ is isopentyl. In some embodiments, R⁷ is 2-methylbutyl. In some embodiments, R⁷ is pentan-2-yl. In some embodiments, R⁷ is 3-methylbutan-2-yl. In some embodiments, R⁷ is pentan-3-yl. In some embodiments, R⁷ is neopentyl. In some embodiments, R⁷ is tert-pentyl. In some embodiments, R⁷ is hexyl. In some embodiments, R⁷ is 4-methylpentyl. In some embodiments, R⁷ is 3-methylpentyl. In some embodiments, R⁷ is 2-methylpentyl. In some embodiments, R⁷ is hexan-2-yl. In some embodiments, R⁷ is 2,3-dimethylbutyl. In some embodiments, R⁷ is 4-methylpentan-2-yl. In some embodiments, R⁷ is 3-methylpentan-2-yl. In some embodiments, R⁷ is 2-ethylbutyl. In some embodiments, R⁷ is hexan-3-yl. In some embodiments, R⁷ is 3,3-dimethylbutyl. In some embodiments, R⁷ is 2,2-dimethylbutyl. In some embodiments, R⁷ is 2-methylpentan-2-yl. In some embodiments, R⁷ is hydrogen.

In some embodiments, at least one R⁸ of formula I is hydroxy. In some embodiments, each R⁸ is hydroxy. In some embodiments, at least one R⁸ is C₁₋₆ hydroxyalkyl. In some embodiments, each R⁸ is C₁₋₆ hydroxyalkyl. In some embodiments, at least one R⁸ is hydroxymethyl. In some embodiments, each R⁸ is hydroxymethyl. In some embodiments, at least one R⁸ is hydroxy and at least one R⁸ is hydroxymethyl. In some embodiments, at least one R⁸ is C₁₋₆ alkyl. In some embodiments, each R⁸ is C₁₋₆ alkyl. In some embodiments, at least one R⁸ is C₁₋₆ alkoxy. In some embodiments, each R⁸ is C₁₋₆ alkoxy. In some embodiments, at least one R⁸ is halogen. In some embodiments, each R⁸ is halogen. In some embodiments, at least one R⁸ is C₁₋₆ haloalkyl. In some embodiments, each R⁸ is C₁₋₆ haloalkyl. In some embodiments, at least one R⁸ is nitro. In some embodiments, each R⁸ is nitro. In some embodiments, at least one R⁸ is cyano. In some embodiments, each R⁸ is cyano.

In some embodiments, at least one R⁹ of formula I is hydroxy. In some embodiments, each R⁹ is hydroxy. In some embodiments, at least one R⁹ is C₁₋₆ hydroxyalkyl. In some embodiments, each R⁹ is C₁₋₆ hydroxyalkyl. In some embodiments, at least one R⁹ is C₁₋₆ alkyl. In some embodiments, each R⁹ is C₁₋₆ alkyl. In some embodiments, at least one R⁹ is C₁₋₆ alkoxy. In some embodiments, each R⁹ is C₁₋₆ alkoxy. In some embodiments, at least one R⁹ is halogen. In some embodiments, each R⁹ is halogen. In some embodiments, at least one R⁹ is C₁₋₆ haloalkyl. In some embodiments, each R⁹ is C₁₋₆ haloalkyl. In some embodiments, at least one R⁹ is nitro. In some embodiments, each R⁹ is nitro. In some embodiments, at least one R⁹ is cyano. In some embodiments, each R⁹ is cyano.

In some embodiments, one of X¹ of formula I and X² of formula I is C and the other of X¹ and X² is Si. In some embodiments, X¹ is C and X² is Si. In some embodiments, X¹ is Si and X² is C. In some embodiments, X¹ is Si and X² is Si.

In some embodiments, subscript m of formula I is an integer from 0 to 3. In some embodiments, m is an integer from 1 to 4. In some embodiments, m is an integer from 0 to 2. In some embodiments, m is an integer from 1 to 3. In some embodiments, m is an integer from 2 to 4. In some embodiments, m is 0. In some embodiments, m is 1. In some embodiments, m is 2. In some embodiments, m is 3. In some embodiments, m is 4.

In some embodiments, subscript n of formula I is an integer from 0 to 8. In some embodiments, n is an integer from 1 to 9. In some embodiments, n is an integer from 0 to 7. In some embodiments, n is an integer from 1 to 8. In some embodiments, n is an integer from 2 to 9. In some embodiments, n is an integer from 0 to 6. In some embodiments, n is an integer from 1 to 7. In some embodiments, n is an integer from 2 to 8. In some embodiments, n is an integer from 3 to 9. In some embodiments, n is an integer from 0 to 5. In some embodiments, n is an integer from 1 to 6. In some embodiments, n is an integer from 2 to 7. In some embodiments, n is an integer from 3 to 8. In some embodiments, n is an integer from 4 to 9. In some embodiments, n is an integer from 0 to 4. In some embodiments, n is an integer from 1 to 5. In some embodiments, n is an integer from 2 to 6. In some embodiments, n is an integer from 3 to 7. In some embodiments, n is an integer from 4 to 8. In some embodiments, n is an integer from 5 to 9. In some embodiments, n is an integer from 0 to 3. In some embodiments, n is an integer from 1 to 4. In some embodiments, n is an integer from 2 to 5. In some embodiments, n is an integer from 3 to 6. In some embodiments, n is an integer from 4 to 7. In some embodiments, n is an integer from 5 to 8. In some embodiments, n is an integer from 6 to 9. In some embodiments, n is an integer from 0 to 2. In some embodiments, n is an integer from 1 to 3. In some embodiments, n is an integer from 2 to 4. In some embodiments, n is an integer from 3 to 5. In some embodiments, n is an integer from 4 to 6. In some embodiments, n is an integer from 5 to 7. In some embodiments, n is an integer from 6 to 8. In some embodiments, n is an integer from 7 to 9. In some embodiments, n is 0. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9.

In some embodiments, the provided compounds of formula I have the structure of formula I(a):

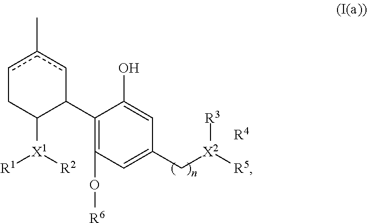

or are a pharmaceutically acceptable salt, solvate, or isomer of a compound with the structure of formula I(a), where R¹, R², R³, R⁴, R⁵, R⁶, X¹, X², and n of formula I(a) are as defined above for formula I.

In some embodiments, the provided compounds of formula I(a) have the structure of formula I(b):

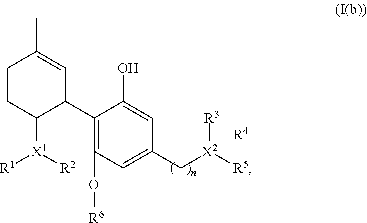

or are a pharmaceutically acceptable salt, solvate, or isomer of a compound with the structure of formula I(b), where R¹, R², R³, R⁴, R⁵, R⁶, X¹, X², and n of formula I(b) are as defined above for formula I.

In some embodiments, the provided compounds of formula I(b) have the structure of formula I(c):

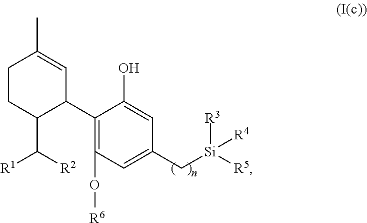

or are a pharmaceutically acceptable salt, solvate, or isomer of a compound with the structure of formula I(c), where R¹, R², R³, R⁴, R⁵, R⁶, and n of formula I(c) are as defined above for formula I.

In some embodiments, the provided compounds of formula I(c) have the structure of formula I(d):

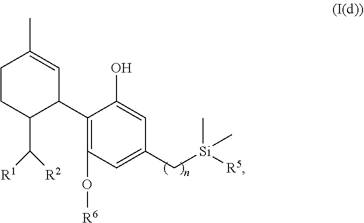

or are a pharmaceutically acceptable salt, solvate, or isomer of a compound with the structure of formula I(d), where R¹, R², R⁵, R⁶, and n of formula I(d) are as defined above for formula I.

In some embodiments, the provided compounds of formula I(d) have the structure of formula I(e):

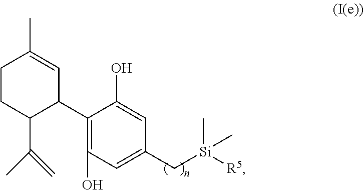

or are a pharmaceutically acceptable salt, solvate, or isomer of a compound with the structure of formula I(e), where R⁵ and n of formula I(e) are as defined above for formula I.

In some embodiments, the provided compounds of formula I(b) have the structure of formula I(f):

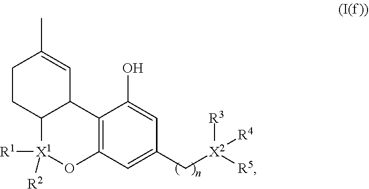

or are a pharmaceutically acceptable salt, solvate, or isomer of a compound with the structure of formula I(f), where R¹, R², R³, R⁴, R⁵, X¹, X², and n of formula I(f) are as defined above for formula I.

In some embodiments, the provided compounds of formula I(f) have the structure of formula I(g):

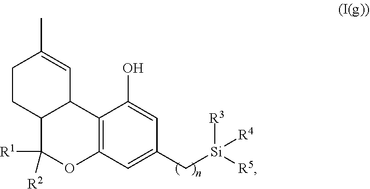

or are a pharmaceutically acceptable salt, solvate, or isomer of a compound with the structure of formula I(g), where R¹, R², R³, R⁴, R⁵, and n of formula I(g) are as defined above for formula I.

In some embodiments, the provided compounds of formula I(g) have the structure of formula I(h):

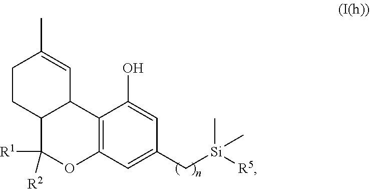

or are a pharmaceutically acceptable salt, solvate, or isomer of a compound with the structure of formula I(h), where R¹, R², R⁵, and n of formula I(h) are as defined above for formula I.

In some embodiments, the provided compounds of formula I(h) have the structure of formula I(i):

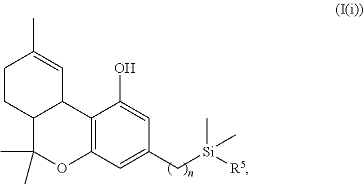

or are a pharmaceutically acceptable salt, solvate, or isomer of a compound with the structure of formula I(i), where R⁵ and n of formula I(i) are as defined above for formula I.

In some embodiments, the provided compounds of formula I(f) have the structure of formula I(j):

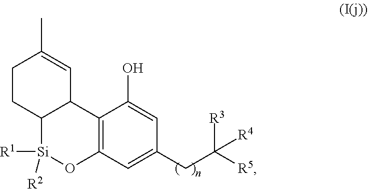

or are a pharmaceutically acceptable salt, solvate, or isomer of a compound with the structure of formula I(j), where R¹, R², R³, R⁴, R⁵ and n of formula I(j) are as defined above for formula I.

In some embodiments, the provided compounds of formula I(j) have the structure of formula I(k):

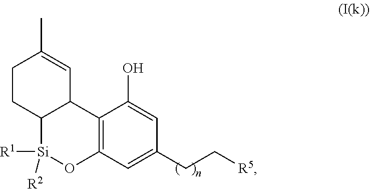

or are a pharmaceutically acceptable salt, solvate, or isomer of a compound with the structure of formula I(k), where R¹, R², R⁵ and n of formula I(k) are as defined above for formula I.

In some embodiments, the provided compounds of formula I(k) have the structure of formula I(l):

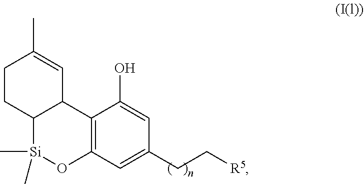

or are a pharmaceutically acceptable salt, solvate, or isomer of a compound with the structure of formula I(l), where R¹, R², R⁵ and n of formula I(l) are as defined above for formula I.

In some embodiments, the provided compound of formula I has the structure:

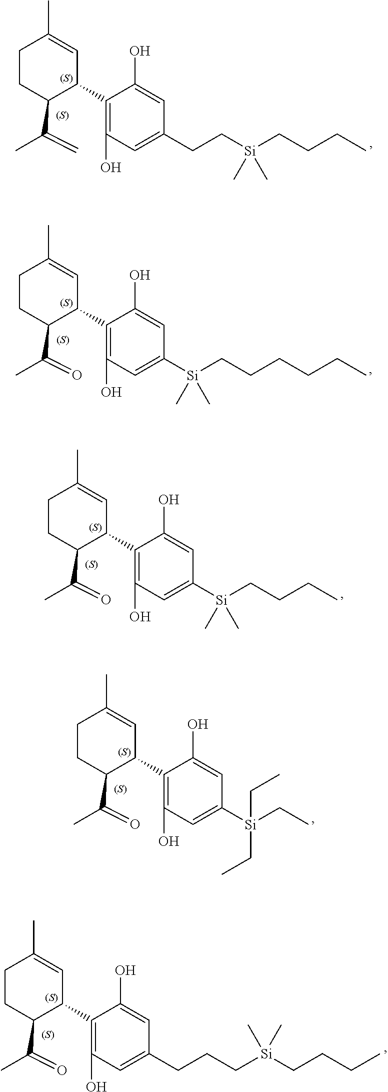

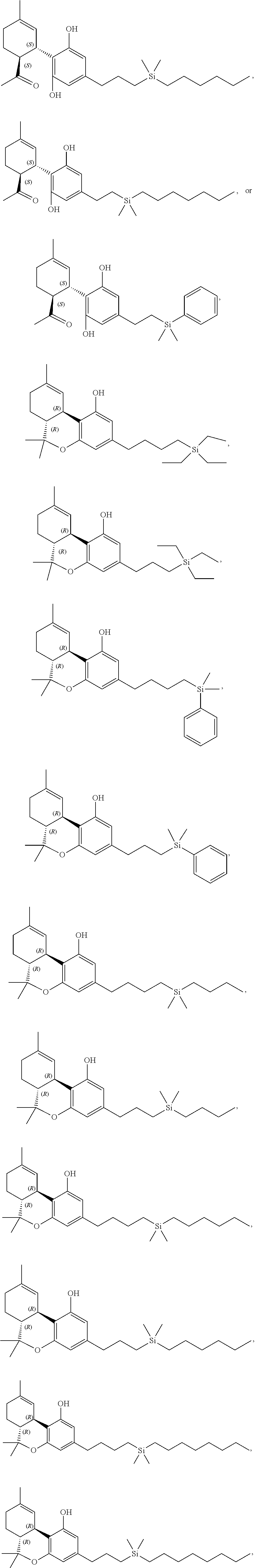

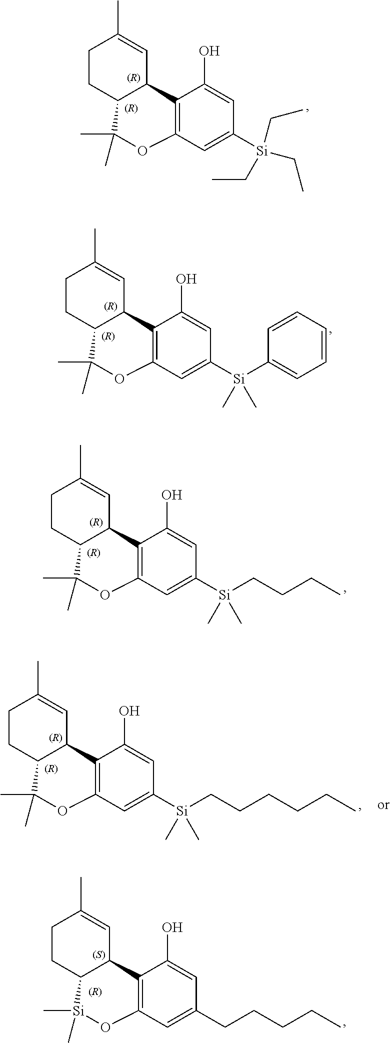

or is a pharmaceutically acceptable salt, solvate, or isomer of a compound with one of the preceding structures.

The compounds provided herein may also be the salts, solvates, and isomers of any on the structures disclosed above. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures), succinates, benzoates, and salts with amino acids such as glutamic acid. These salts may be prepared by methods known to those skilled in art. When compounds of the present disclosure contain relatively basic functionalities, acid addition salts may be obtained by contacting the neutral form of such compounds with a sufficient amount of the desired acid, either neat or in a suitable inert solvent. Examples of acceptable acid addition salts include those derived from inorganic acids such as hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids such as acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like.

Also included are salts of amino acids such as arginate and the like, and salts of organic acids such as glucuronic or galactunoric acids and the like (see, for example, Berge et al., Journal of Pharmaceutical Science 66, (1977): 1). Certain specific compounds of the present disclosure contain basic acidic functionalities that allow the compounds to be converted into base addition salts. Additional information on suitable pharmaceutically acceptable salts can be found in Remington's Pharmaceutical Sciences, 17th ed., Mack Publishing Company, Easton, Pa., 1985, which is incorporated herein by reference.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present disclosure may exist in unsolvated forms as well as solvated forms, including hydrated forms. In general, the solvated forms are equivalent to unsolvated forms and are encompassed within the scope of the present disclosure. Certain compounds of the present invention may exist in multiple crystalline or amorphous forms. In general, all physical forms are equivalent for the uses contemplated by the present disclosure and are intended to be within the scope of the present disclosure.

Certain compounds of the present disclosure possess asymmetric carbon atoms (optical centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those which are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques.

Unless otherwise stated, the compounds of the present disclosure may also contain unnatural proportions of atomic isotopes at one or more of the atoms that constitute such compounds. For example, the compounds of the present disclosure may be radiolabeled with radioactive isotopes, such as for example deuterium (²H), tritium (³H), iodine-125 (¹²⁵I), carbon-13 (¹³C), or carbon-14 (¹⁴C). All isotopic variations of the compounds of the present disclosure, whether radioactive or not, are encompassed within the scope of the present invention.

The present invention further includes compounds which are in a prodrug form. Prodrugs of the compounds described herein are those compounds that readily undergo chemical changes under physiological conditions to provide any of the compounds disclosed above. Additionally, prodrugs can be converted to the compounds of the present disclosure by chemical or biochemical methods in an ex vivo environment. For example, prodrugs can be slowly converted to the compounds of the present invention when placed in a transdermal patch reservoir with a suitable enzyme or chemical reagent.

A surprising advantage of the provided compounds is that they may have beneficially strong agonist activity towards at least one of cannabinoid receptor type 2 (CB₂) or cannabinoid receptor type 1 (CB₁). A strong agonist activity is desirable because it indicates that the provided compound may be therapeutically effective in treating disorders related to the cannabinoid receptor. For example, a strong agonist activity may indicate potency useful for treating neurological disorders and/or providing neuroprotective properties. In some embodiments, the provided compound has strong agonist activity towards CB₂. In some embodiments, the provided compound has strong agonist activity towards CB₁. In some embodiments, the provided compound has strong agonist activity towards both CB₂ and CB₁. The compound may exhibit agonist activity towards CB₂, CB₁, or CB₂ and CB₁ with an EC₅₀ that is, for example, between 10 nM and 1000 nM, e.g., between 16 nM and 250 nM, between 25 nM and 400 nM, between 40 nM and 630 nM, or between 63 nM and 1000 nM. In terms of upper limits, the compound may have an agonist activity towards CB₂, CB₁, or CB₂ and CB₁ with an EC₅₀ that is, for example, less than 1000 nM, e.g., less than 630 nM, less than 400 nM, less than 250 nM, less than 160 nM, less than 100 nM, less than 63 nM, less than 40 nM, less than 25 nM, or less than 16 nM. Stronger agonist activities, e.g., those with an EC₅₀ less than 10 nM, are also contemplated.

Another surprising advantage of the provided compounds is that they have may have beneficially highly selective activity towards one of CB₁ and CB₂ relative to the other of CB₁ and CB₂. A high selectivity is desirable because it indicates that the provided compound may, for example, offer an effective treatment for neurological disorders and/or inflammation while simultaneously avoiding unwanted psychoactive effects. The selectivity of a provided compound may be such that, for example, the compound exhibits an agonist activity towards CB₂ relative to that towards CB₁ that is between 50-fold and 1000-fold greater, e.g., between 50-fold and 300-fold greater, between 67-fold and 410-fold greater, between 91-fold and 450-fold greater, between 120-fold and 740-fold greater, or between 170-fold and 1000-fold greater. In terms of lower limits, the compound may exhibit an agonist activity towards CB₂ relative to an agonist activity towards CB₁ that is, for example, at least 50-fold greater, e.g., at least 67-fold greater, at least 91-fold greater, at least 120-fold greater, at least 170-fold greater, at least 220-fold greater, at least 300-fold greater, at least 410-fold greater, at least 550-fold greater, or at least 740-fold greater. Higher selectivities, e.g, those for which the agonist activity towards CB₂ is at least 1000-fold greater than the agonist activity towards CB₁, are also contemplated. Also contemplated are selectivities for which the agonist activity towards CB₁ is greater than the agonist activity towards CB₂ by any of the magnitudes disclosed herein.

### IV. Pharmaceutical Compositions

In another aspect, the present disclosure provides pharmaceutical compositions including one or more pharmaceutically acceptable carriers, diluents, excipients, or buffers and one or more of the compounds provided herein. In some embodiments, the pharmaceutically acceptable carrier, diluent, excipient, or buffer is suitable for use in a subject, for example, a human. Pharmaceutically acceptable excipients include, but are not limited to, liquids such as water, saline, glycerol, sugars and ethanol. The preparation of pharmaceutically acceptable carriers and excipients is described in, e.g., Remington: The Science and Practice of Pharmacy, 22nd edition, Loyd V. Allen et al, editors, Pharmaceutical Press (2012).

In some embodiments, the composition also includes an additional active compound or other chemotherapeutic agent. In some embodiments, the pharmaceutical composition further includes one or more stabilizing compounds, which may be administered in any sterile, biocompatible pharmaceutical carrier, including, but not limited to, saline, buffered saline, dextrose, and water. In some embodiments, the pharmaceutical compositions also contain a pharmaceutically acceptable salt. Pharmaceutically acceptable salts can include, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in the provided pharmaceutical compositions. The pharmaceutical compositions may be subjected to conventional pharmaceutical operations such as sterilization.

### V. Formulations

The compositions provided herein can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by a subject, e.g., a human patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol. 35, (1995): 1187-1193; Tjwa, Ann. Allergy Asthma Immunol. 75, (1995): 107).

For preparing pharmaceutical formulations including the compounds of the present disclosure, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature.

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% by weight of the compounds of the present disclosure.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention may also be used orally using, for example, push-fit capsules made of gelatin, as well as soft, sealed capsules made of gelatin and a coating such as glycerol or sorbitol. Push-fit capsules may contain the compounds of the present invention mixed with a filler or binders such as lactose or starches, lubricants such as talc or magnesium stearate, and, optionally, stabilizers. In soft capsules, the compounds of the present invention may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compounds of the present disclosure are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations may be formulated in solution in aqueous polyethylene glycol solution.

Aqueous solutions suitable for oral use can be prepared by dissolving the compounds of the present invention in water and adding suitable colorants, flavors, stabilizers, and thickening agents as desired. Aqueous suspensions suitable for oral use may be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension may also contain one or more preservatives such as ethyl or n-propyl p-hydroxybenzoate, one or more coloring agents, one or more flavoring agents and one or more sweetening agents, such as sucrose, aspartame or saccharin. Formulations may be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions may be formulated by suspending the compounds of the present invention in a vegetable oil, such as arachis oil, olive oil, sesame oil, or coconut oil; or in a mineral oil such as liquid paraffin; or a mixture of these. The oil suspensions may contain a thickening agent, such as beeswax, hard paraffin, or cetyl alcohol. Sweetening agents, such as glycerol, sorbitol or sucrose, may be added to provide a palatable oral preparation. These formulations may be preserved by the addition of an antioxidant such as ascorbic acid. As an example of an injectable oil vehicle, see Minto, J. Pharmacol. Exp. Ther. 281, (1997): 93. The pharmaceutical formulations of the disclosure may also be in the form of oil-in-water emulsions. The oily phase may be a vegetable oil or a mineral oil, described above, or a mixture of these. Suitable emulsifying agents include naturally occurring gums such as gum acacia and gum tragacanth, naturally occurring phosphatides such as soybean lecithin, esters or partial esters derived from fatty acids and hexitol anhydrides such as sorbitan mono-oleate, and condensation products of these partial esters with ethylene oxide such as polyoxyethylene sorbitan mono-oleate. The emulsion may also contain sweetening agents and flavoring agents, as in the formulation of syrups and elixirs. Such formulations may also contain a demulcent, a preservative, or a coloring agent.

The compositions of the present invention may also be delivered as microspheres for slow release in the body. For example, microspheres may be formulated for administration via intradermal injection of drug-containing microspheres, which slowly release subcutaneously (see Rao, J. Biomater Sci. Polym. Ed. 7, (1995): 623); as biodegradable and injectable gel formulations (see, e.g., Gao Pharm. Res. 12, (1995): 857); or as microspheres for oral administration (see, e.g., Eyles, J. Pharm. Pharmacol. 49, (1997): 669). Both transdermal and intradermal routes may afford constant delivery for weeks or months.

In other embodiments, the compositions of the present disclosure are formulated for parenteral administration, such as intravenous (IV) administration or administration into a body cavity or lumen of an organ. Such formulations for administration will commonly comprise a solution of the compositions of the present disclosure dissolved in a pharmaceutically acceptable carrier. Among the acceptable vehicles and solvents that may be employed are water and Ringer's solution, an isotonic sodium chloride. In addition, sterile fixed oils may conventionally be employed as a solvent or suspending medium. For this purpose, any bland fixed oil may can be employed including synthetic mono- or diglycerides. In addition, fatty acids such as oleic acid may likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH-adjusting and buffering agents, toxicity adjusting agents, e.g., sodium acetate, sodium chloride, potassium chloride, calcium chloride, sodium lactate and the like. The concentration of the compositions of the present disclosure in these formulations may vary widely, and will be selected primarily based on fluid volumes, viscosities, body weight, and the like, in accordance with the particular mode of administration selected and the patient's needs. For IV administration, the formulation may be a sterile injectable preparation, such as a sterile injectable aqueous or oleaginous suspension. This suspension may be formulated according to the known art using those suitable dispersing or wetting agents and suspending agents. The sterile injectable preparation may also be a sterile injectable solution or suspension in a nontoxic parenterally acceptable diluent or solvent, such as a solution of 1,3-butanediol.

In another embodiment, formulations of the compositions of the present disclosure are delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, delivery may be focused into the target cells in vivo. (See, e.g., Al-Muhammed, J. Microencapsul. 13, (1996): 293; Chonn, Curr. Opin. Biotechnol. 6, (1995): 698; Ostro, Am. J. Hosp. Pharm. 46, (1989): 1576-1587).

Lipid-based formulations include lipid solutions, lipid emulsions, lipid dispersions, self-emulsifying drug delivery systems (SEDDS) and self-microemulsifying drug delivery systems (SMEDDS). In particular, SEDDS and SMEDDS are isotropic mixtures of lipids, surfactants and co-surfactants that can disperse spontaneously in aqueous media and form fine emulsions (SEDDS) or microemulsions (SMEDDS). Lipids useful in the formulations of the present disclosure include any natural or synthetic lipids including, but not limited to, sesame seed oil, olive oil, castor oil, peanut oil, fatty acid esters, glycerol esters, LABRAFIL®, LABRASOL®, CREMOPHOR®, SOLUTOL®, TWEEN®, CAPRYOL®, CAPMUL®, CAPTEX®, and PECEOL®.

### VI. Methods of Preparing Compounds

The present disclosure also provides many synthetic routes and methods for preparing the compounds disclosed herein. In some embodiments, the method is for preparing compounds having the structure of formula I(m):

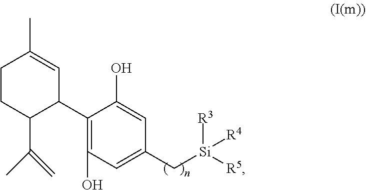

or are a pharmaceutically acceptable salt, solvate, or isomer of a compound with the structure of formula I(m). R³ and R⁴ of formula I(m) can each independently be hydrogen, C₁₋₆ alkyl, ═CH₂, or C₂₋₆ alkenyl, or can be combined with X² to form a 3- to 12-membered heterocyclyl. R⁵ of formula I(m) can be hydrogen, C₁₋₉ alkyl, ═CH₂, C₂₋₆ alkenyl, C₆₋₁₂ aryl, or C₁₋₆ alkyl-C₆₋₁₂ aryl, where the aryl and the alkyl-aryl are optionally substituted with 1-4 R⁹ groups. Each R⁹ can independently be C₁₋₆ alkyl, hydroxy, C₁₋₆ alkoxy, C₁₋₆ hydroxyalkyl, halogen, C₁₋₆ haloalkyl, nitro, or cyano. Subscript n of formula I(m) can be an integer from 0 to 9. The method includes forming a silyl cannabinoid synthesis reaction mixture comprising 1-methyl-4-(prop-1-en-2-yl) cyclohex-2-en-1-ol and a compound of formula II:

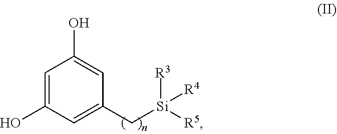

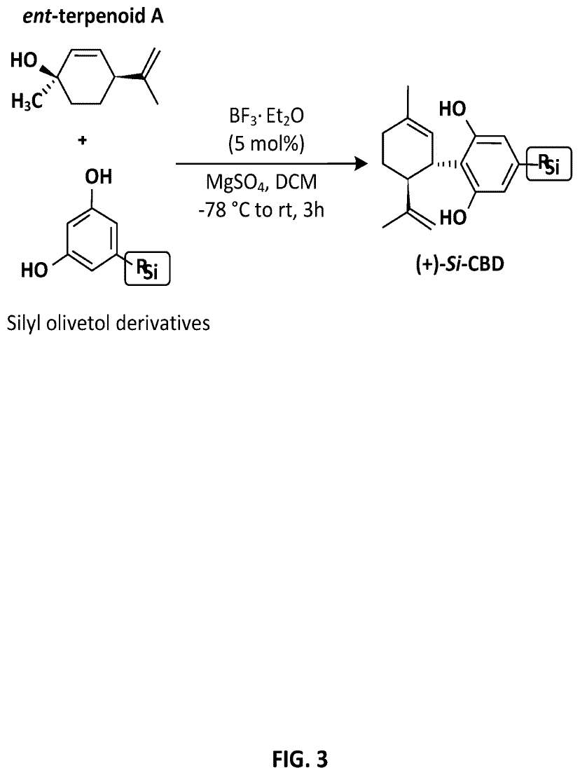

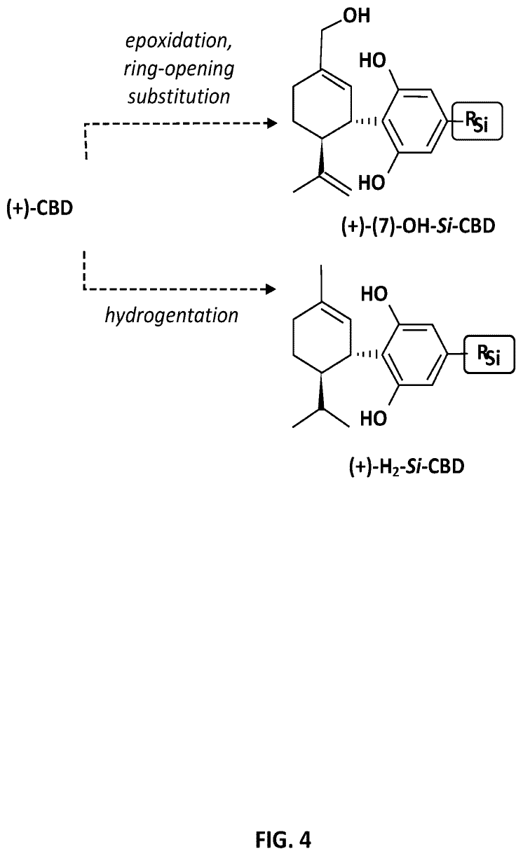

under conditions sufficient to form the compound of formula I(m). FIG. 3 illustrates an exemplary reaction scheme in accordance with the provided method. FIG. 4 illustrates synthesis of additional provided compounds

In some embodiments, R³ of formula I(m) is C₁₋₆ alkyl. In some embodiments, R³ is methyl. In some embodiments, R³ is ethyl. In some embodiments, R³ is propyl. In some embodiments, R³ is isopropyl. In some embodiments, R³ is butyl. In some embodiments, R³ is isobutyl. In some embodiments, R³ is sec-butyl. In some embodiments, R³ is tert-butyl. In some embodiments, R³ is pentyl. In some embodiments, R³ is isopentyl. In some embodiments, R³ is 2-methylbutyl. In some embodiments, R³ is pentan-2-yl. In some embodiments, R³ is 3-methylbutan-2-yl. In some embodiments, R³ is pentan-3-yl. In some embodiments, R³ is neopentyl. In some embodiments, R³ is tert-pentyl. In some embodiments, R³ is hexyl. In some embodiments, R³ is 4-methylpentyl. In some embodiments, R³ is 3-methylpentyl. In some embodiments, R³ is 2-methylpentyl. In some embodiments, R³ is hexan-2-yl. In some embodiments, R³ is 2,3-dimethylbutyl. In some embodiments, R³ is 4-methylpentan-2-yl. In some embodiments, R³ is 3-methylpentan-2-yl. In some embodiments, R³ is 2-ethylbutyl. In some embodiments, R³ is hexan-3-yl. In some embodiments, R³ is 3,3-dimethylbutyl. In some embodiments, R³ is 2,2-dimethylbutyl. In some embodiments, R³ is 2-methylpentan-2-yl. In some embodiments, R³ is hydrogen. In some embodiments, R³ is ═CH₂. In some embodiments, R³ is C₂₋₆ alkenyl.

In some embodiments, R⁴ of formula I(m) is C₁₋₆ alkyl. In some embodiments, R⁴ is methyl. In some embodiments, R⁴ is ethyl. In some embodiments, R⁴ is propyl. In some embodiments, R⁴ is isopropyl. In some embodiments, R⁴ is butyl. In some embodiments, R⁴ is isobutyl. In some embodiments, R⁴ is sec-butyl. In some embodiments, R⁴ is tert-butyl. In some embodiments, R⁴ is pentyl. In some embodiments, R⁴ is isopentyl. In some embodiments, R⁴ is 2-methylbutyl. In some embodiments, R⁴ is pentan-2-yl. In some embodiments, R⁴ is 3-methylbutan-2-yl. In some embodiments, R⁴ is pentan-3-yl. In some embodiments, R⁴ is neopentyl. In some embodiments, R⁴ is tert-pentyl. In some embodiments, R⁴ is hexyl. In some embodiments, R⁴ is 4-methylpentyl. In some embodiments, R⁴ is 3-methylpentyl. In some embodiments, R⁴ is 2-methylpentyl. In some embodiments, R⁴ is hexan-2-yl. In some embodiments, R⁴ is 2,3-dimethylbutyl. In some embodiments, R⁴ is 4-methylpentan-2-yl. In some embodiments, R⁴ is 3-methylpentan-2-yl. In some embodiments, R⁴ is 2-ethylbutyl. In some embodiments, R⁴ is hexan-3-yl. In some embodiments, R⁴ is 3,3-dimethylbutyl. In some embodiments, R⁴ is 2,2-dimethylbutyl. In some embodiments, R⁴ is 2-methylpentan-2-yl. In some embodiments, R⁴ is hydrogen. In some embodiments, R⁴ is-CH₂. In some embodiments, R⁴ is C₂₋₆ alkenyl.

In some embodiments, R⁵ of formula I(m) is C₁₋₆ alkyl. In some embodiments, R⁵ is methyl. In some embodiments, R⁵ is ethyl. In some embodiments, R⁵ is propyl. In some embodiments, R⁵ is isopropyl. In some embodiments, R⁵ is butyl. In some embodiments, R⁵ is isobutyl. In some embodiments, R⁵ is sec-butyl. In some embodiments, R⁵ is tert-butyl. In some embodiments, R⁵ is pentyl. In some embodiments, R⁵ is isopentyl. In some embodiments, R⁵ is 2-methylbutyl. In some embodiments, R⁵ is pentan-2-yl. In some embodiments, R⁵ is 3-methylbutan-2-yl. In some embodiments, R⁵ is pentan-3-yl. In some embodiments, R⁵ is neopentyl. In some embodiments, R⁵ is tert-pentyl. In some embodiments, R⁵ is hexyl. In some embodiments, R⁵ is 4-methylpentyl. In some embodiments, R⁵ is 3-methylpentyl. In some embodiments, R⁵ is 2-methylpentyl. In some embodiments, R⁵ is hexan-2-yl. In some embodiments, R⁵ is 2,3-dimethylbutyl. In some embodiments, R⁵ is 4-methylpentan-2-yl. In some embodiments, R⁵ is 3-methylpentan-2-yl. In some embodiments, R⁵ is 2-ethylbutyl. In some embodiments, R⁵ is hexan-3-yl. In some embodiments, R⁵ is 3,3-dimethylbutyl. In some embodiments, R⁵ is 2,2-dimethylbutyl. In some embodiments, R⁵ is 2-methylpentan-2-yl. In some embodiments, R⁵ is any of the aforementioned C₁₋₆ alkyl groups substituted with 1 to 4 R⁹ groups.

In some embodiments, R⁵ of formula I(m) is C₆₋₁₂ aryl. In some embodiments, R⁵ is C₆₋₁₂ phenyl. In some embodiments, R⁵ is naphthyl. In some embodiments, R⁵ is biphenyl. In some embodiments, R⁵ is acenaphthyl. In some embodiments, R⁵ is fluorenyl. In some embodiments, R⁵ is indenyl. In some embodiments, R⁵ is azulenyl. In some embodiments, R⁵ is hydrogen. In some embodiments, R⁵ is —CH₂. In some embodiments, R⁵ is C₂₋₆ alkenyl. In some embodiments, R⁵ is C₁₋₆ alkyl-C₆₋₁₂ aryl. In some embodiments, R⁵ is any of the aforementioned aryl, alkenyl, or alkyl-aryl groups substituted with 1 to 4 R⁹ groups.

In some embodiments, subscript n of formula I(m) is an integer from 0 to 8. In some embodiments, n is an integer from 1 to 9. In some embodiments, n is an integer from 0 to 7. In some embodiments, n is an integer from 1 to 8. In some embodiments, n is an integer from 2 to 9. In some embodiments, n is an integer from 0 to 6. In some embodiments, n is an integer from 1 to 7. In some embodiments, n is an integer from 2 to 8. In some embodiments, n is an integer from 3 to 9. In some embodiments, n is an integer from 0 to 5. In some embodiments, n is an integer from 1 to 6. In some embodiments, n is an integer from 2 to 7. In some embodiments, n is an integer from 3 to 8. In some embodiments, n is an integer from 4 to 9. In some embodiments, n is an integer from 0 to 4. In some embodiments, n is an integer from 1 to 5. In some embodiments, n is an integer from 2 to 6. In some embodiments, n is an integer from 3 to 7. In some embodiments, n is an integer from 4 to 8. In some embodiments, n is an integer from 5 to 9. In some embodiments, n is an integer from 0 to 3. In some embodiments, n is an integer from 1 to 4. In some embodiments, n is an integer from 2 to 5. In some embodiments, n is an integer from 3 to 6. In some embodiments, n is an integer from 4 to 7. In some embodiments, n is an integer from 5 to 8. In some embodiments, n is an integer from 6 to 9. In some embodiments, n is an integer from 0 to 2. In some embodiments, n is an integer from 1 to 3. In some embodiments, n is an integer from 2 to 4. In some embodiments, n is an integer from 3 to 5. In some embodiments, n is an integer from 4 to 6. In some embodiments, n is an integer from 5 to 7. In some embodiments, n is an integer from 6 to 8. In some embodiments, n is an integer from 7 to 9. In some embodiments, n is 0. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9.

In some embodiments, R³ of formula I(m) and R⁴ of formula I(m) are each methyl, and subscript n of formula I(m) is 0. In some embodiments, the provided method further includes forming a silyl olivetol synthesis reaction mixture comprising 1-bromo-3,5-dimethoxybenzene and a compound of formula III:

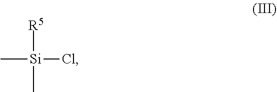

under conditions sufficient to form the compound of formula IV:

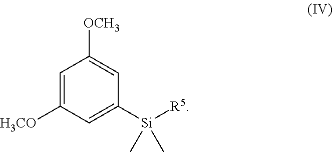

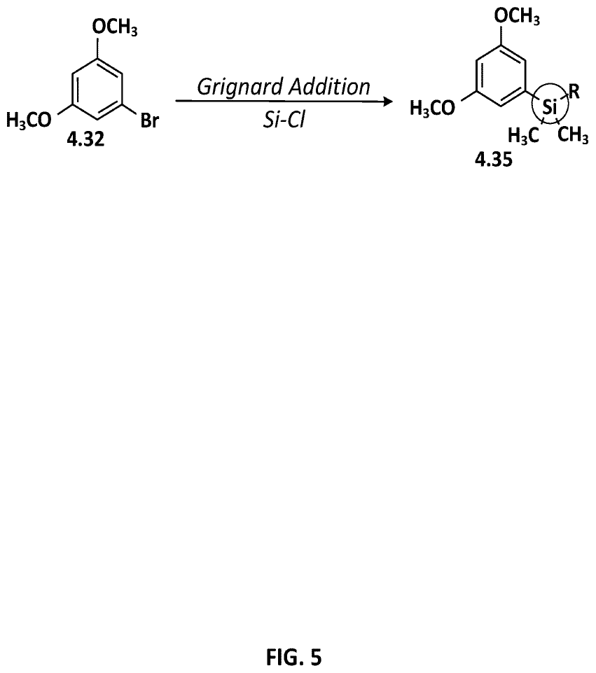

FIG. 5 illustrates an exemplary reaction scheme in accordance with the provided method.

In some embodiments, R³ of formula I(m) and R⁴ of formula I(m) are each methyl, and subscript n of formula I(m) is an integer from 3 to 9. In some embodiments, the provided method further includes forming a silyl olivetol synthesis reaction mixture comprising a compound of formula V:

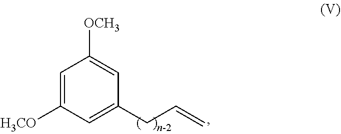

and a compound of formula VI:

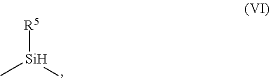

under conditions sufficient to form the compound of formula VII:

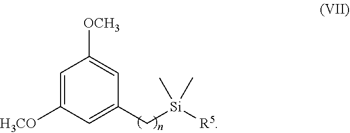

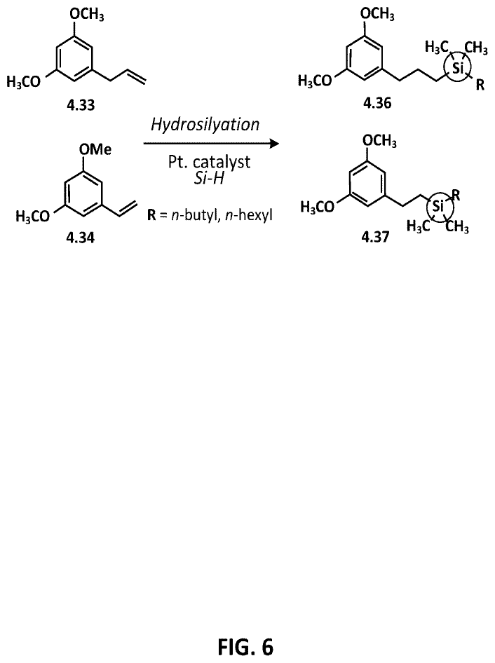

FIG. 6 illustrates exemplary reaction schemes in accordance with the provided method.

Each of the preceding reaction mixtures for the provided methods for preparing the compounds may further include one or more catalysts. In some embodiments, the provided method further includes selecting the one or more catalysts based on desired yields, specificities, and/or purities, e.g., isomeric purities, of the reaction products. In some embodiments, a metal hydrosilylation catalyst may be used. In some embodiments, a platinum hydrosilylation catalyst may be used. For example, the platinum hydrosilylation catalyst may be a platinum oxide catalyst or a Karstedt catalyst.

### VII. Methods of Treating a Disorder

In another aspect, the present disclosure provides a method of treating a disorder. The method includes administering to a subject in need of such a treatment a therapeutically effective amount of a compound disclosed herein, thereby treating the disorder. In some embodiments, the treatment is given with a curative intent. In some embodiments, the treatment is given with an aim to prolong the life of the subject. In some embodiments, the treatment is given for the purpose of reducing symptoms associated with the disorder. In some embodiments, symptoms reduced by the treatment include seizures, loss of consciousness, convulsions, muscle spasms, staring spells, confusion, hallucinations, changes in mood or behavior, difficulty speaking, or any combination thereof. In some embodiments, the treatment is given prophylactically to a subject, e.g., a subject with a family history of epilepsy, a subject suffering a head injury or brain damage, a subject infected with meningitis or encephalitis, a subject with autism or Down syndrome, a subject suffering from a stroke, a subject with a brain tumor, or a subject suffering from Alzheimer's disease, such that the subject is considered to be at an elevated risk of developing a disorder or symptoms typically associated with a disorder.

In some embodiments, the disorder is a neurodegenerative disorder. The neurodegenerative disorder may be, for example and without limitation, Alzheimer's disease, Parkinson's disease, Huntington's disease, amyotrophic lateral sclerosis (ALS), multiple sclerosis (MS), a prion disease, or frontotemporal dementia (FTD). In some embodiments, the disorder includes epilepsy. In some embodiments, the disorder includes inflammation.

### VIII. Exemplary Embodiments

The following embodiments are contemplated. All combinations of features and embodiments are contemplated.

Embodiment 1: A compound having the formula:

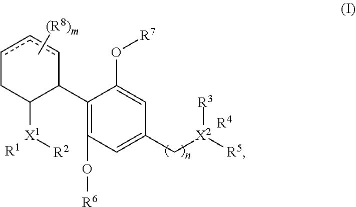

or a pharmaceutically acceptable salt, solvate, or isomer thereof, wherein R¹ and R² are each independently selected from the group consisting of hydrogen, C₁₋₆ alkyl, ═CH₂, and C₂₋₆ alkenyl; R³ and R⁴ are each independently selected from the group consisting of hydrogen, C₁₋₆ alkyl, ═CH₂, and C₂₋₆ alkenyl, or are combined with X² to form a 3- to 12-membered heterocyclyl; R⁵ is selected from the group consisting of hydrogen, C₁₋₉ alkyl, ═CH₂, C₂₋₆ alkenyl, C₆₋₁₂ aryl, and C₁₋₆ alkyl-C₆₋₁₂ aryl, wherein the aryl and the alkyl-aryl are optionally substituted with 1-4 R⁹ groups; R⁶ is selected from the group consisting of hydrogen, C₁₋₆ alkyl, and a bond connected to X¹; R⁷ is selected from the group consisting of hydrogen and C₁₋₆ alkyl; each R⁸ and R⁹ is independently selected from the group consisting of C₁₋₆ alkyl, hydroxy, C₁₋₆ alkoxy, C₁₋₆ hydroxyalkyl, halogen, C₁₋₆ haloalkyl, nitro, and cyano; X¹ and X² are each independently selected from the group consisting of C and Si, with the proviso that at least one of X¹ and X² is Si; the subscript m is an integer from 0 to 4; and the subscript n is an integer from 0 to 9.

Embodiment 2: An embodiment of embodiment 1, wherein R¹ and R² are each independently selected from the group consisting of C₁₋₆ alkyl and —CH₂.

Embodiment 3: An embodiment of embodiment 2, wherein R¹ and R² are each independently methyl or ═CH₂.

Embodiment 4: An embodiment of any of embodiment 1-3, wherein R³ and R⁴ are each independently C₁₋₆ alkyl.

Embodiment 5: An embodiment of embodiment 4, wherein each R³ and R⁴ are each independently methyl or ethyl.

Embodiment 6: An embodiment of any of embodiment 1-5, wherein R⁵ is selected from the group consisting of C₁₋₈ alky and C₆₋₁₂ aryl.

Embodiment 7: An embodiment of embodiment 6, wherein R⁵ is selected from the group consisting of ethyl, butyl, hexyl, phenyl, and octyl.

Embodiment 8: An embodiment of any of embodiment 1-7, wherein R⁶ is selected from the group consisting of methyl and a bond connected to X¹.

Embodiment 9: An embodiment of any of embodiment 1-8, wherein R⁷ is hydrogen.

Embodiment 10: An embodiment of any of embodiment 1-9, wherein each R⁸ is selected from the group consisting of hydroxy and C₁₋₆ hydroxyalkyl.

Embodiment 11: An embodiment of embodiment 10, wherein each R⁸ is selected from the group consisting of hydroxy and hydroxymethyl.

Embodiment 12: An embodiment of any of embodiment 1-11, wherein one of X¹ and X² is C.

Embodiment 13: An embodiment of any of embodiment 1-12, wherein m is 1.

Embodiment 14: An embodiment of any of embodiment 1-13, wherein n is an integer from 0 to 4.

Embodiment 15: An embodiment of embodiment 1, having the formula:

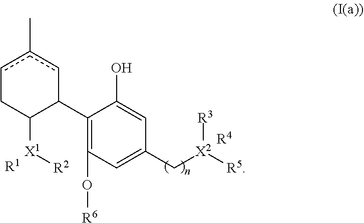

Embodiment 16: An embodiment of embodiment 15, having the formula:

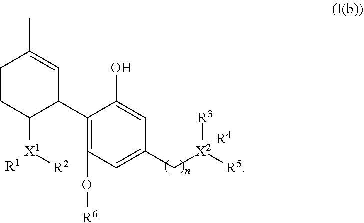

Embodiment 17: An embodiment of embodiment 16, having the formula:

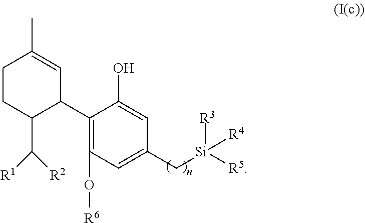

Embodiment 18: An embodiment of embodiment 17, having the formula:

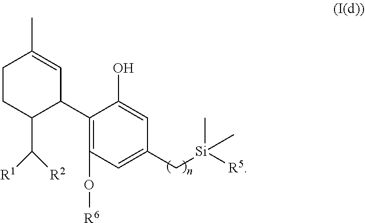

Embodiment 19: An embodiment of embodiment 18, having the formula:

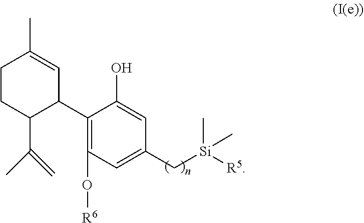

Embodiment 20: An embodiment of embodiment 19, wherein the compound is selected from the group consisting of:

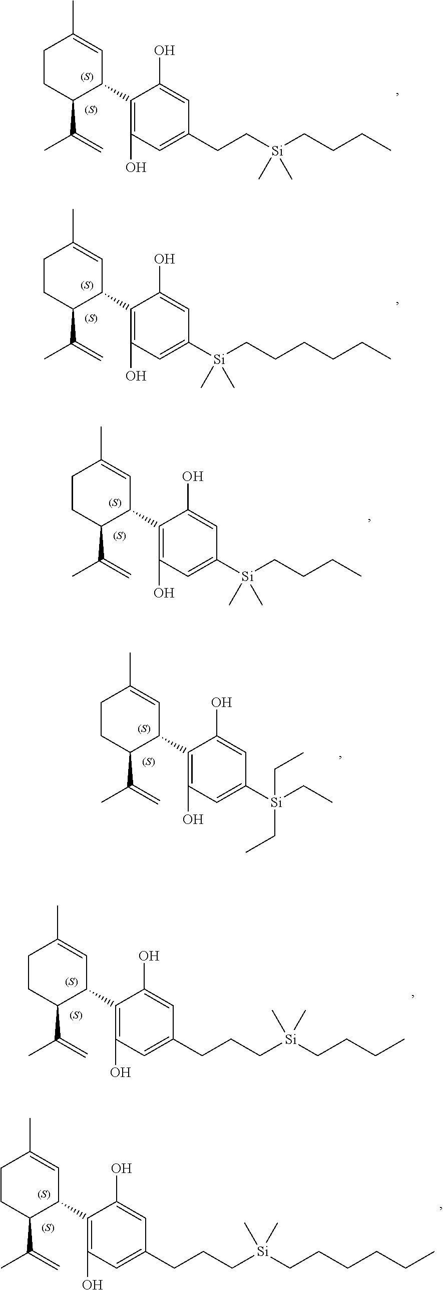

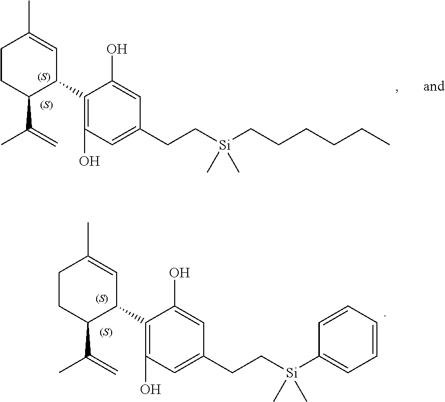

Embodiment 21: An embodiment of embodiment 16, having the formula:

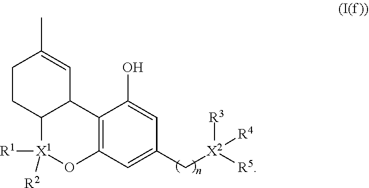

Embodiment 22: An embodiment of embodiment 21, having the formula:

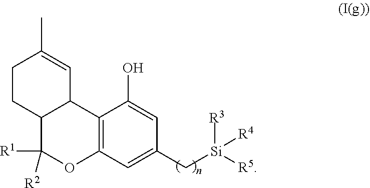

Embodiment 23: An embodiment of embodiment 22, having the formula:

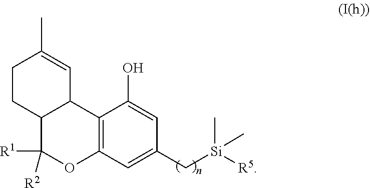

Embodiment 24: An embodiment of embodiment 23, having the formula:

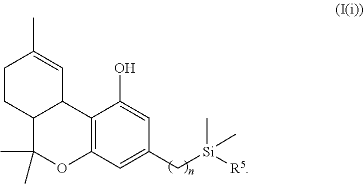

Embodiment 25: An embodiment of embodiment 24, wherein the compound is selected from the group consisting of:

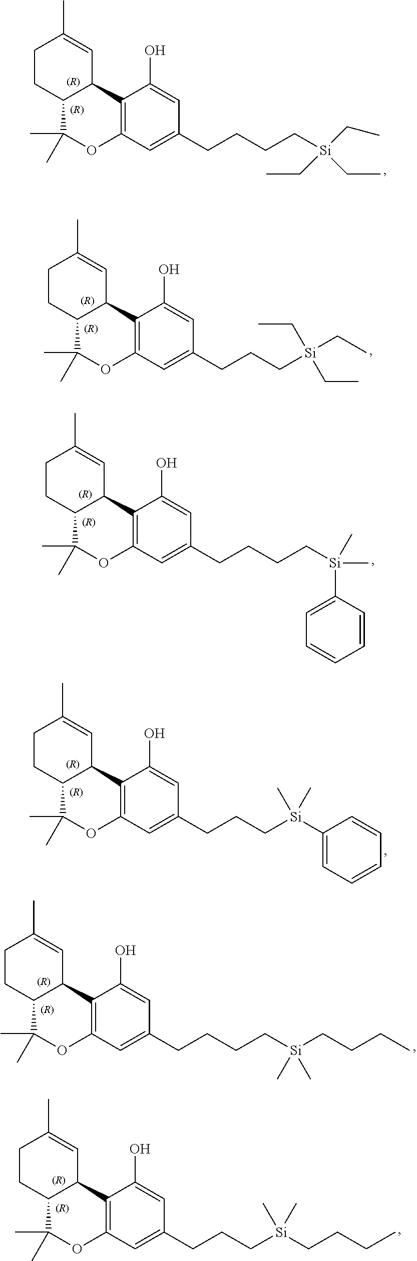

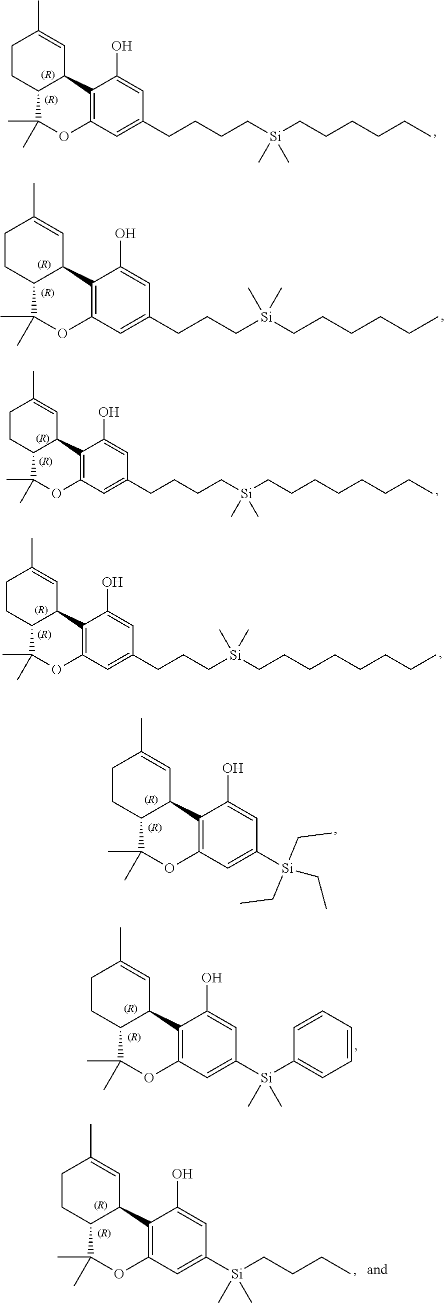

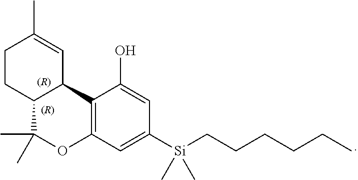

Embodiment 26: An embodiment of embodiment 21, having the formula:

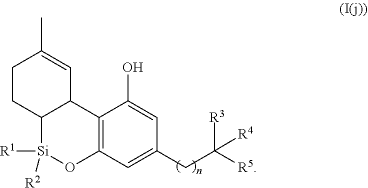

Embodiment 27: An embodiment of embodiment 26, having the formula:

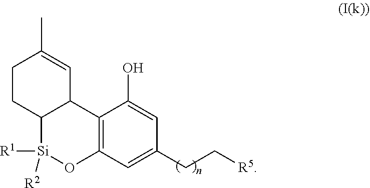

Embodiment 28: An embodiment of embodiment 27, having the formula:

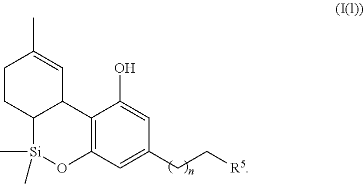

Embodiment 29: An embodiment of embodiment 28, wherein the compound is:

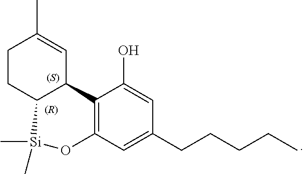

Embodiment 30: An embodiment of any of embodiments 1-29, wherein the compound exhibits agonist activity with an EC₅₀ of less than 1000 nM towards at least one of cannabinoid receptor type 2 (CB₂) or cannabinoid receptor type 1 (CB₁).

Embodiment 31: An embodiment of any of embodiments 1-30, wherein the compound exhibits agonist activity towards CB₂ that is at least 50-fold greater than agonist activity towards CB₁.

Embodiment 32: A method of preparing a compound having the structure:

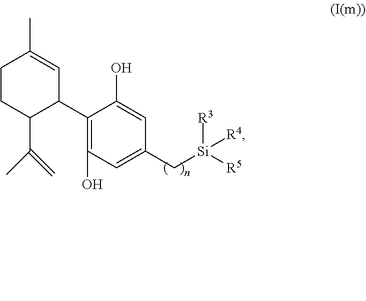

the method comprising: forming a silyl cannabinoid synthesis reaction mixture comprising 1-methyl-4-(prop-1-en-2-yl) cyclohex-2-en-1-ol and a compound of formula II:

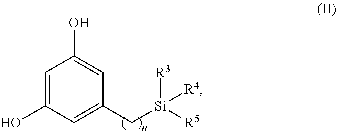

under conditions sufficient to form the compound of formula I(m), wherein R³ and R⁴ are each independently selected from the group consisting of hydrogen, C₁₋₆ alkyl, ═CH₂, and C₂₋₆ alkenyl, or are combined with X² to form a 3- to 12-membered heterocyclyl; R⁵ is selected from the group consisting of hydrogen, C₁₋₉ alkyl, ═CH₂, C₂₋₆ alkenyl, C₆₋₁₂ aryl, and C₁₋₆ alkyl-C₆₋₁₂ aryl, wherein the aryl and the alkyl-aryl are optionally substituted with 1-4 R⁹ groups; each R⁹ is independently selected from the group consisting of C₁₋₆ alkyl, hydroxy, C₁₋₆ alkoxy, C₁₋₆ hydroxyalkyl, halogen, C₁₋₆ haloalkyl, nitro, and cyano; and the subscript n is an integer from 0 to 9.

Embodiment 34: An embodiment of embodiment 33, wherein R³ and R⁴ are each methyl; n is 0; and the method further comprises: forming a silyl olivetol synthesis reaction mixture comprising 1-bromo-3,5-dimethoxybenzene and a compound of formula III:

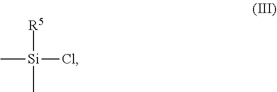

under conditions sufficient to form the compound of formula IV:

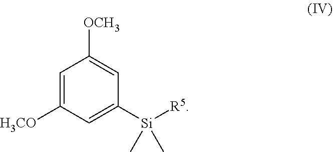

Embodiment 35: An embodiment of embodiment 33, wherein R³ and R⁴ are each methyl; n is from 3 to 9; and the method further comprises: forming a silyl olivetol synthesis reaction mixture comprising a compound of formula V:

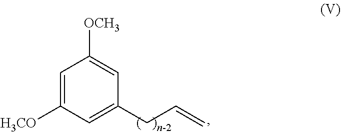

and a compound of formula VI:

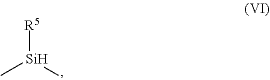

under conditions sufficient to form the compound of formula VII:

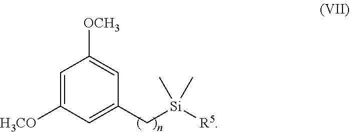

Embodiment 36: A method of treating a disorder, the method comprising administering to a subject in need thereof a therapeutically effective amount of the compound of an embodiment of any of embodiments 1-29 or the pharmaceutical composition of embodiment 32.

Embodiment 37: An embodiment of embodiment 36, wherein the disorder is a neurological disorder.

Embodiment 38: An embodiment of embodiment 37, wherein the disorder is a neurodegenerative disorder.

Embodiment 39: An embodiment of embodiment 37 or 38, wherein the disorder comprises epilepsy.

Embodiment 40: An embodiment of any of embodiments 36-39, wherein the disorder comprises inflammation.

### EXAMPLES

The present disclosure will be better understood in view of the following non-limiting examples. The following examples are intended for illustrative purposes only and do not limit in any way the scope of the present invention

### Example 1. In Vitro Evaluation of Potency and CB₂ Selectivity of Silyl-CBD Compounds

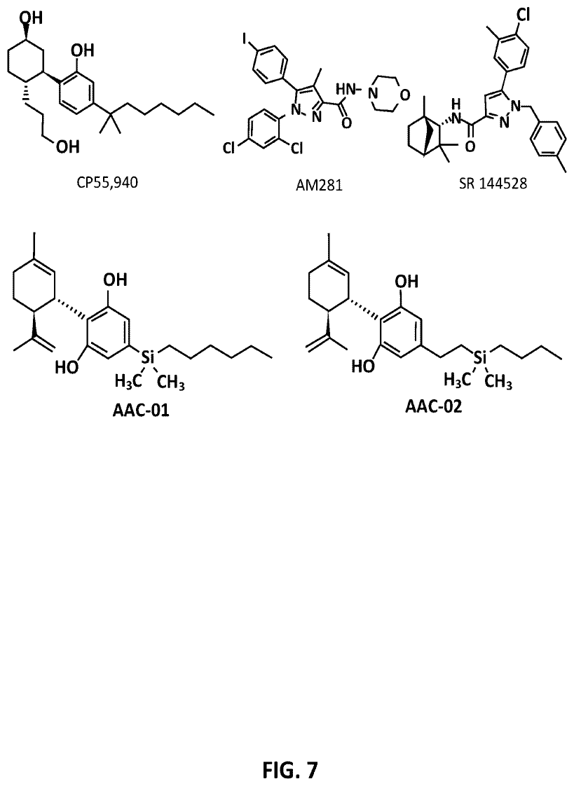

Silyl-CBD compounds are evaluated using Eurofin's CB₁ and CB₂ β-arrestin recruitment agonist/antagonist assay to gain insight in the structural features responsible for selectivity and potency for the CB₂ receptor. The provided compounds are profiled with four biosensor assays against three compounds: CP55,940, a known potent agonist of CB₁ and CB₂ receptors with Ki values of 0.58 nM and 0.68 nM, respectively; AM281, a selective CB₁ receptor antagonist; and SR 144528, a CB₂ receptor inverse agonist (FIG. 7). The RC₅₀ data of these potent agonist and antagonist for the CB_{1/2} receptors serve as a controls for comparing the activity of the provided silyl-CBD compounds.

The plasma matrix stability profile of each of the provided compounds is tested utilizing LC-MS/MS sample analysis in a non-compartmental analysis (NCA) of rat plasma. Oral and intravenous dosing is used to determine parameters including variation of drug concentration in plasma over time (AUCₗₐₛₜ, AUC_{INF}), the average lifetime of the drug in a matrix (mean resident time, MRT), mean terminal half-life (t_{1/2}), the dose-response levels in the form of maximum concentration of the drug in the matrix (Cₘₐₓ), and time to maximum plasma concentration (Tₘₐₓ). Metabolism is assessed using a microsomal stability assay to measure in vitro intrinsic clearance and profile metabolites formed. A cross-species microsomal stability identifies potential differences in metabolism which can assist in interpreting pharmacology and toxicity data. Caco-2 cell permeability assays are also conducted to demonstrate improved membrane permeation. The Caco-2 monolayer is widely used across the pharmaceutical industry as an in vitro model of the human small intestinal mucosa to predict the absorption of orally administered drugs.

Results from the β-arrestin recruitment agonist/antagonist assay of two of the provided compounds, designated AAC-01 and AAC-02 (FIG. 7), are presented in Table 1 below. For antagonist assays, data was normalized to the maximal and minimal response observed in the presence of EC₈₀ ligand and vehicle. The following EC₅₀ concentrations were used: CB₁ Arrestin: 0.016 μM, CP55940 CB₂ Arrestin: 0.0053 μM CP55940. As shown in the data of the table, AAC-02 displays no agonist or antagonist activity towards CB₁, exhibiting an EC₅₀ of >10,000 nM and an IC₅₀ of 2229.5 nM, respectively. AAC-02 does, however, have strong agonist activity towards the CB₂ receptor, exhibiting an EC₅₀ of 18.0 nM. This superb 500× selectivity for CB₂ vs CB₁ is rarely observed in non-classical cannabinoid analogs. Notably, AAC-01 exhibits no CB₂ vs CB₁ selectivity, but has potent agonist activity towards these receptors, with an EC₅₀ of 4.14 nM and 33.3 nM, respectively. These are comparable to the CB_{1/2} agonist activity of CP55,940. The demonstrated selectivity is the result of the design strategy disclosed herein, as AAC-02 only differs from AAC-01 based on the provided strategic placement of the silyl group.

TABLE 1
Biological assay data for AAC-01 and AAC-02
as compared to CP55,940, AM281 and SR 144528.

| Compound | Assay | Target | Result | RC₅₀ (nM) | Eₘₐₓ (%) |
|---|---|---|---|---|---|
| CP55,940 | agonist | CB₁ | EC₅₀ | 6.35 | 104.1 |
| AM281 | antagonist | CB₁ | IC₅₀ | 10.47 | 106.6 |
| CP55,940 | agonist | CB₂ | EC₅₀ | 2.09 | 100.6 |
| SR 144528 | antagonist | CB₂ | IC₅₀ | 6.08 | 101.6 |
| AAC-01 | agonist | CB₁ | EC₅₀ | 33.3 | 64.8 |
| AAC-01 | antagonist | CB₁ | IC₅₀ | 2065.1 | 86.4 |
| AAC-01 | agonist | CB₂ | EC₅₀ | 4.14 | 70.0 |
| AAC-01 | antagonist | CB₂ | IC₅₀ | 1871.0 | 69.7 |
| AAC-02 | agonist | CB₁ | EC₅₀ | >10,000 | — |
| AAC-02 | antagonist | CB₁ | IC₅₀ | 2229.5 | 71.1 |
| AAC-02 | agonist | CB₂ | EC₅₀ | 18.03 | 50.3 |
| AAC-02 | antagonist | CB₂ | IC₅₀ | >10,000 | — |

### Example 2. Evaluation of Silyl-CBD Efficacy of in Rat Models of Neuroinflammation

Rat data is used to demonstrate that the provided silyl-CBD compounds are neuroprotective and anti-inflammatory in response to a model of an induced surge of neurodegeneration with slow ongoing cell death and chronic inflammation. Rats are treated with pilocarpine (350 mg/kg) to induce neurodegeneration vs a vehicle control and then treated with a provided compound or with vehicle. Duration and severity of seizures are quantified over the next four hours, with outcomes compared against the pilocarpine-vehicle controls. The rats are perfused after two weeks. Tissue is sectioned and then labeled with antibodies for both neurons (NeuN) and microglia (Iba-1). Using stereology, the number of neurons is quantified as well as the number and morphology of Iba-1 to demonstrate a reduction in cell death as compared to pilocarpine-vehicle controls.

### Example 3. Cardiac Safety Assessment of Silyl-CBD Compounds

A comprehensive in vitro proarrythmia study is performed to measure adverse cardiotoxicity interactions, including inhibition of the hERG human potassium ion channel, the Nav1.5 human sodium ion channel, and Cav12 (L-type) human calcium ion channel. The hERG-lite assay predicts types of hERG risk early in the drug development process. The cardiac safety panel includes ion channel screening with patch clamp electrophysiology studies to measure in vitro blockage of ion channels. Considering CBD's mechanism of action in the brain and central nervous system, cytotoxicity is determined by dose-response curves in primary forebrain neuronal cells in a high throughput screening assay using high content imaging systems for unbiased image acquisition and analysis.

All patents, patent publications, patent applications, journal articles, books, technical references, and the like discussed in the instant disclosure are incorporated herein by reference in their entirety for all purposes.

It is to be understood that the figures and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments of the disclosure, such substitution is considered within the scope of the disclosure.

The examples presented herein are intended to illustrate potential and specific implementations of the disclosure. It can be appreciated that the examples are intended primarily for purposes of illustration of the disclosure for those skilled in the art. There may be variations to these diagrams or the operations described herein without departing from the spirit of the disclosure. For instance, in certain cases, method steps or operations may be performed or executed in differing order, or operations may be added, deleted or modified.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

In the foregoing description, numerous specific details are set forth to provide a more thorough understanding of the present invention. However, it will be apparent to one of skill in the art that the invention described in this disclosure may be practiced without one or more of these specific details. In other instances, well-known features and procedures well known to those skilled in the art have not been described in order to avoid obscuring the invention. Embodiments of the disclosure have been described for illustrative and not restrictive purposes. Although the present invention is described primarily with reference to specific embodiments, it is also envisioned that other embodiments will become apparent to those skilled in the art upon reading the present disclosure, and it is intended that such embodiments be contained within the present inventive methods. Accordingly, the present disclosure is not limited to the embodiments described above or depicted in the drawings, and various embodiments and modifications can be made without departing from the scope of the claims below.

1. A compound having the formula: 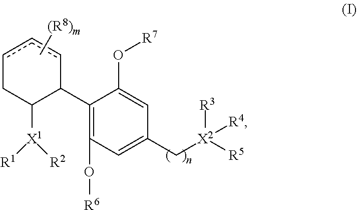
or a pharmaceutically acceptable salt, solvate, or isomer thereof, wherein
R¹ and R² are each independently selected from the group consisting of hydrogen, C₁₋₆ alkyl, ═CH₂, and C₂₋₆ alkenyl;
R³ and R⁴ are each independently selected from the group consisting of hydrogen, C₁₋₆ alkyl, ═CH₂, and C₂₋₆ alkenyl, or are combined with X² to form a 3- to 12-membered heterocyclyl;
R⁵ is selected from the group consisting of hydrogen, C₁₋₉ alkyl, ═CH₂, C₂₋₆ alkenyl, C₆₋₁₂ aryl, and C₁₋₆ alkyl-C₆₋₁₂ aryl, wherein the aryl and the alkyl-aryl are optionally substituted with 1-4 R⁹ groups;
R⁶ is selected from the group consisting of hydrogen, C₁₋₆ alkyl, and a bond connected to X¹;
R⁷ is selected from the group consisting of hydrogen and C₁₋₆ alkyl;
each R⁸ and R⁹ is independently selected from the group consisting of C₁₋₆ alkyl, hydroxy, C₁₋₆ alkoxy, C₁₋₆ hydroxyalkyl, halogen, C₁₋₆ haloalkyl, nitro, and cyano;
X¹ and X² are each independently selected from the group consisting of C and Si, with the proviso that at least one of X¹ and X² is Si;
the subscript m is an integer from 0 to 4; and
the subscript n is an integer from 0 to 9.

2. The compound of claim 1, wherein R¹ and R² are each independently selected from the group consisting of C₁₋₆ alkyl and ═CH₂.

3. The compound of claim 1, wherein R³ and R⁴ are each independently C₁₋₆ alkyl.

4. The compound of claim 1, wherein R⁵ is selected from the group consisting of C₁₋₈ alky and C₆₋₁₂ aryl.

5. The compound of claim 1, wherein R⁶ is selected from the group consisting of methyl and a bond connected to X¹.

6. The compound of claim 1, wherein R⁷ is hydrogen.

7. The compound of claim 1, wherein each R⁸ is selected from the group consisting of hydroxy and C₁₋₆ hydroxyalkyl.

8. The compound of claim 1, wherein one of X¹ and X² is C.

9. The compound of claim 1, wherein m is 1.

10. The compound of claim 1, wherein n is an integer from 0 to 4.

11. The compound of claim 1, wherein the compound is selected from the group consisting of:

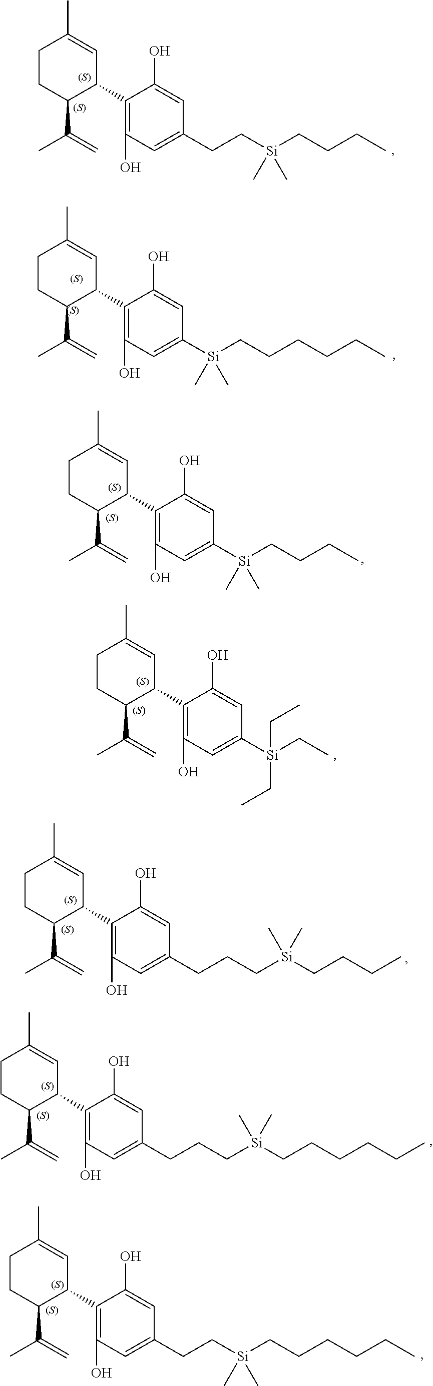

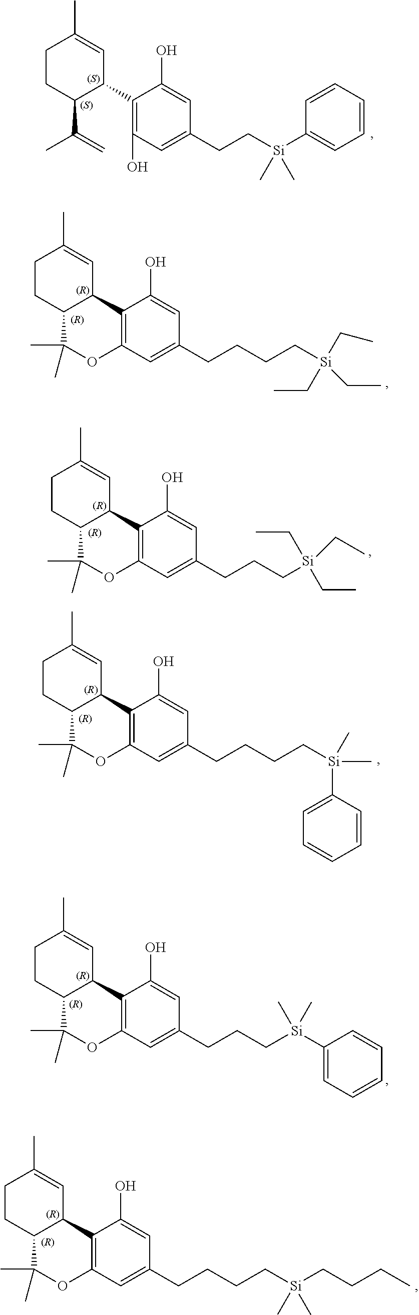

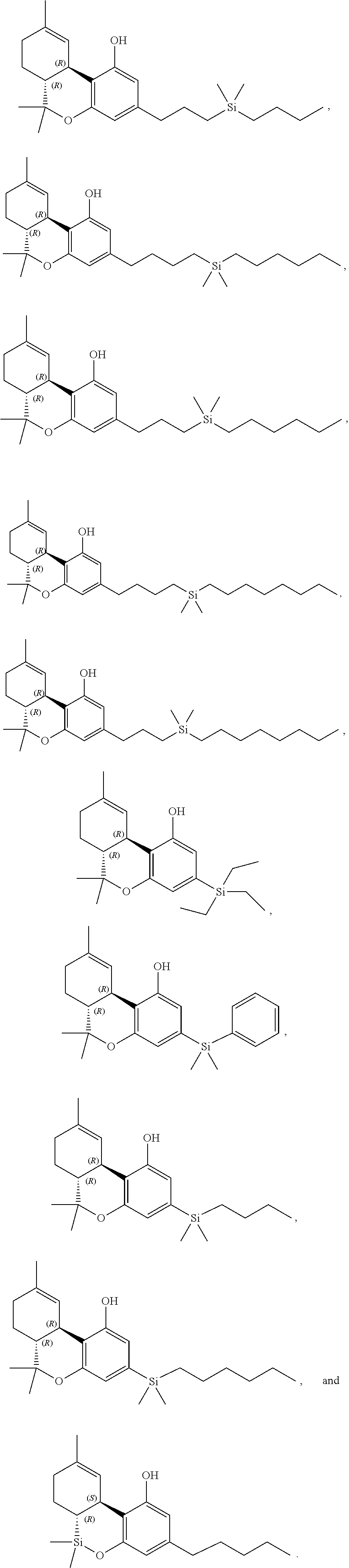

12. The compound of claim 1, wherein the compound exhibits agonist activity with an EC₅₀ of less than 1000 nM towards at least one of cannabinoid receptor type 2 (CB₂) or cannabinoid receptor type 1 (CB₁).

13. The compound of claim 1, wherein the compound exhibits agonist activity towards CB₂ that is at least 50-fold greater than agonist activity towards CB₁.

14. A pharmaceutical composition comprising the compound of claim 1 and a pharmaceutically acceptable carrier.

15. A method of preparing a compound having the structure: 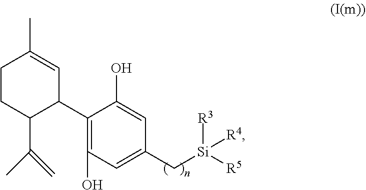
the method comprising:
forming a silyl cannabinoid synthesis reaction mixture comprising 1-methyl-4-(prop-1-en-2-yl) cyclohex-2-en-1-ol and a compound of formula II: 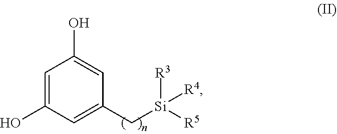
under conditions sufficient to form the compound of formula I(m), wherein
R³ and R⁴ are each independently selected from the group consisting of hydrogen, C₁₋₆ alkyl, ═CH₂, and C₂₋₆ alkenyl, or are combined with X² to form a 3- to 12-membered heterocyclyl;
R⁵ is selected from the group consisting of hydrogen, C₁₋₉ alkyl, ═CH₂, C₂₋₆ alkenyl, C₆₋₁₂ aryl, and C₁₋₆ alkyl-C₆₋₁₂ aryl, wherein the aryl and the alkyl-aryl are optionally substituted with 1-4 R⁹ groups;
each R⁹ is independently selected from the group consisting of C₁₋₆ alkyl, hydroxy, C₁₋₆ alkoxy, C₁₋₆ hydroxyalkyl, halogen, C₁₋₆ haloalkyl, nitro, and cyano; and
the subscript n is an integer from 0 to 9.

16. The method of claim 15, wherein
R³ and R⁴ are each methyl;
n is 0; and
the method further comprises:
forming a silyl olivetol synthesis reaction mixture comprising 1-bromo-3,5-dimethoxybenzene and a compound of formula III: 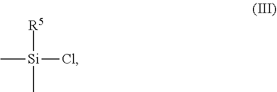
under conditions sufficient to form the compound of formula IV:

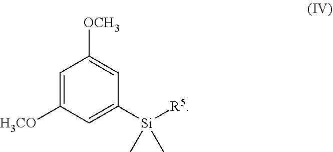

17. The method of claim 15, wherein
R³ and R⁴ are each methyl;
n is from 3 to 9; and
the method further comprises:
forming a silyl olivetol synthesis reaction mixture comprising a compound of formula V: 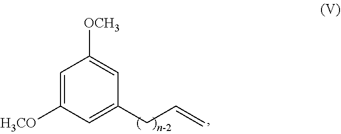
and
a compound of formula VI: 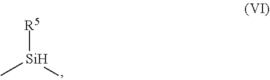
under conditions sufficient to form the compound of formula VII:

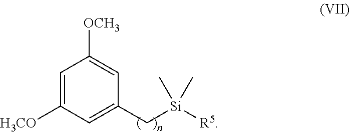

18. A method of treating a disorder, the method comprising administering to a subject in need thereof a therapeutically effective amount of the compound of claim 1.

19. The method of claim 18, wherein the disorder is a neurological disorder.

20. The method of claim 18, wherein the disorder comprises epilepsy.
